(19) United States
(12) Patent Application Publication
(10) Pub. No.: US 2003/0082101 A1
(43) Pub. Date: May 1, 2003

(54) Accelerators for increasing the rate of formation of free radicals and reactive oxygen species

(75) Inventors: Kevin Taylor, Mason, OH (US); Jody Mesaros, Mason, OH (US)

Correspondence Address: BROWDY AND NEIMARK, P.L.L.C., 624 Ninth Street, N.W., Washington, DC, 20001, US

(73) Assignee: Cavalier Discovery, Mason, OH

(21) Appl. No.: 10/166,038

(22) Filed: Jun. 11, 2002

Related U.S. Application Data
(60) Provisional application No. 60/296,761, filed on Jun. 11, 2001.

(51) Int. Cl. A61K 51/00; A61K 31/555; A61K 33/24

(52) U.S. Cl. 424/1.11; 514/185; 514/58; 424/617; 604/20

(57) ABSTRACT

The formation of free radicals is enhanced with photodynamic agents, sonodynamic agents, and systems and therapies utilizing ultrasound by subjecting the agent to light waves or sound waves in the presence of a metal, a reductant, or a chelate, or mixtures thereof.

### CROSS REFERENCE TO RELATED APPLICATION

The present application claims priority from non-provisional Application Serial No. 60/296,761, filed Jun. 11, 2001, the entire contents of which are hereby incorporated.

### FIELD OF THE INVENTION

This invention relates to methods and compositions which can increase the effectiveness of therapies and processes which involve chemical reactions which produce radicals and reactive oxygen species.

Such therapies and processes include, but are not limited to, sonodynamic therapy, high intensity focused ultrasound (HIFU) therapies, photodynamic therapy, radiation therapy for cancer treatment, chemotherapy, waste water treatment, treatment of contaminated soil with ultrasound, sterilization with ultrasound, and polymerization reactions facilitated by ultrasound. Therapies and processes which utilize ultrasound are particularly well-suited to this invention.

### BACKGROUND OF THE INVENTION

Photodynamic therapy (PDT) involves the use of photosensitizable compounds for selective destruction of biological tissue, such as tumors, using a photosensitizable drug which may be linked to a tumor-localizing agent such as an antibody, followed by exposure of the target region to light. Photosensitizable compounds are molecules that are activated by light of a characteristic wavelength, usually from a laser, ultimately resulting in the formation of cytotoxic intermediates such as singlet oxygen or free radicals. The photosensitizable compound acts either at the cell surface, or is internalized, ultimately destroying the membrane at the cell surface or on cellular organella, respectively, leading to cell death. In cancer treatment the tumor destruction is believed to proceed via one or both of the following two suggested mechanisms: the intravascular pathway, i.e., collapse of blood vessels with which hamper blood perfusion to the tumor and deprive the tumor of oxygen and nutrients; and/or the parenchymal tumor pathways in with which the tumor is destroyed by direct necrotic effects on the tumor cells. One of the severe problems with photodynamic therapy is post-treatment sensitivity to sunlight, which required that the patients remain out of direct light for several weeks after photosensitizable compounds have been administered.

Sonodynamic therapy (SDT) is relatively newer than photodynamic therapy, and is based upon the synergistic effect of drugs and ultrasound in producing cytotoxic effects on tissues, particularly on tumors. The cytotoxicity of SDT can be enhanced by the presence of sonosensitizable compounds, i.e., agents with which can emit single oxygen or free radicals in response to irradiation by ultrasound. Some photosensitive compounds, such as porphyrin and porphryinyl analogs, have been found to be sonosensitizable agents in cultures of tumor cells. A problem with some sonodynamic therapies is that the sonodynamic agent is cytotoxic in the absence of ultrasound.

Ultrasonic cavitation (the ultrasound-driven growth of microbubbles from tiny gas pockets present in a solution, and their subsequent violent collapse which produces locally extreme temperatures and pressures inside these collapsible bubbles) seems to be required for a sonodynamic effect. Although the mechanism of sonosensitization is not understood, it appears that reactive radical intermediates formed from these compounds by ultrasound, either as a result of direct pyrolysis in the hot cavitation bubbles or after reaction with the OH radicals and H atoms which are produced by sonnolysis of water, are involved in cell killing. Formation of peroxyl radicals from DMF and DMSO has been demonstrated in highly diluted air-saturated solutions of these compounds exposed to 50 kHz ultrasound.

### SUMMARY OF THE INVENTION

It is an object of the present invention to overcome the aforementioned deficiencies in the prior art.

It is another object of the present invention to achieve increased rates of free radical production from aforementioned therapies and processes.

It is another object of the present invention to achieve increased rates of free radical production from sonodynamic and photodynamic therapy systems.

It is another object of the present invention to provide a new class of sonodynamic therapy agents.

It is a further object of the present invention to increase the rate of formation of cytotoxic species from existing sonodynamic systems and agents by combination with the disclosed agents.

It is a further object of the present invention to provide improved methods for treating patients using sonodynamic or photodynamic therapy.

It is yet another object of the present invention to achieve increased rates of free radical production from sonodynamic and photodynamic systems.

It is still another object of the present invention to provide a method for combining sonodynamic therapy and photodynamic therapy to enhance the effects of both therapies.

According to the present invention, the rate of formation of free radicals from sonodynamic and photodynamic systems can be increased by adding at least one activator which can be a transition metal, a reducing agent (reductant), or a transition metal chelator (chelant) to a photodynamic or sonodynamic agent prior to irradiating with the appropriate exogenous energy. This method can be used to increase the formation of free radicals in chemical or biological systems, including in production of polymers, wastewater or soil treatment, treatment of patients, etc.

The addition of an accelerator, which is at least one of a transition meal, a reductant, or a chelant, provides faster free radical production as well as enhanced radical production via the addition of more chemical pathways which generate radicals.

A transition metal chelating compound can be added to the combination of metal and reductant to further accelerate the production of toxic free radicals by lowering the redox potential of the metal allowing the metal to react more easily. These chelating compounds also promote production of free radicals by maintaining iron in a soluble form.

The present invention is able to take advantage of the Fenton and Fenton-type reactions, which involves the reaction of hydrogen peroxide with a transition metal to produce hydroxyl radical and hydroxyl radical ion. According to the present invention, ultrasound can be used to accelerate the Fenton reaction in vivo.

Bicarbonate ion can be added to the compounds claimed in the patent to further stimulate radical production. Related reference: Stadtman, E. R. Fenton chemistry. The Journal of Biological Chemistry. Vol 266 pp 17201-17211 (1991). The bicarbonate can be any bicarbonate salt that produces bicarbonate ion in the reaction medium, including alkali metal bicarbonates, ammonium bicarbonates, etc.

The present invention is able to take further advantage of Fenton and Fenton-type reactions by adding an additional transition metal, adding a chelant, and/or adding a reductant. The chelant preferably reduces the reduction potential of the transition metal, and the reductant preferably has a reduction potential which permits reduction of the transition metal or transition metal complex to a lower oxidation number. Free radical production is also promoted by the chelating compounds, which maintain the iron in a soluble form.

The present invention takes advantage of the correlation between known Fenton activity of a substance and the ability to accelerate free radical production during exposure to ultrasound. For example, a compound that exhibits Fenton activity in an enzymatic system or a radiolytic system is also able to accelerate radical production in an ultrasound system of the present invention.

The effectiveness of existing sonodynamic drugs can be improved by taking advantage of the Fenton reaction by adding more transition metal, adding a chelant, and/or adding a reductant to reduce the metal.

A new sonodynamic drug is presented where a reductant such as ascorbic acid is added to the diseased tissues. Upon application of ultrasound, iron from biological sources is mobilized and interacts with hydrogen peroxide generated from the action of ultrasound on water and oxygen, resulting in the production of hydroxyl ion and hydroxyl ion radical. The reductant accelerates the reaction of the metal by reducing it back to an active species after it has reacted with the hydrogen peroxide.

A new sonodynamic drug is presented where a quinone or a quinone containing species is added to the diseased tissues. The quinone containing species interacts with ultrasound to form semiquinone radical, and the semiquinone radical acts as a transition metal reductant. Upon application of ultrasound, iron from biological sources is mobilized and will interact with hydrogen peroxide generated from the action of ultrasound on water and oxygen, resulting in the production of hydroxyl ion and hydroxyl ion radical. The reductant accelerates the reaction of the metal by reducing it back to an active species after it has reacted with the hydrogen peroxide.

A new sonodynamic drug is presented where a quinone or a quinone containing species is added to the diseased tissues. The quinone containing species interacts with ultrasound to form semiquinone radical, and the semiquinone radical mobilizes transition metals such as iron from biological sources. Upon application of ultrasound, iron interacts with hydrogen peroxide generated from the action of ultrasound on water and oxygen, resulting in the production of hydroxyl ion and hydroxyl ion radical. The semiquinone radical then serves as a reductant which accelerates the reaction of the metal by reducing it back to an active species after it has reacted with the hydrogen peroxide.

Quinone compounds can also accelerate radical productions by:

1. chelating iron

2. generating superoxide by redox cycling, and

3. releasing iron from biological sources.

A new sonodynamic drug is presented where a chelant is added to the diseased tissues. Upon application of ultrasound, iron from biological sources is mobilized and will interact with hydrogen peroxide generated from the action of ultrasound on water and oxygen, resulting in the production of hydroxyl ion and hydroxyl ion radical. The chelant, for example EDTA, accelerates the reaction of the metal by reducing its redox potential and allowing it to react more easily with hydrogen peroxide, and/or by chelating the oxidized metal and maintaining it in a state that can be reduced back to an active form of the metal, for example oxalate. Additionally, the chelating compounds promote production of free radicals by maintaining iron in a soluble form.

Compounds that stimulate the production of hydrogen peroxide in the body can be used along with the process of the present invention to enhance free radical production. Examples of these substances include but are not limited to 3-amino-1,2,4-triazole; 6-formylpterin; sinuline; systemin; methyl jasmonate; thrombin; substance P; sn-1,2-dioctanoylglycerol; ionomycin; formylmethionyl-leucyl-phenylalanine; interferon gamma; poly-L-histidine; and 6-hydroxydopamine.

Macrophage/Neutrophil stimulators can be used along with the ultrasound process of the present invention to enhance production of free radicals. Examples of these stimulators include but are not limited to polysaccharides such as sizofiran, fucosamine and krebiozen; leucokinins such as tuftsin; granulocyte-macrophage colony-stimulating factors such as Regramostim, Sargramostim, Milodistim, Molgramostin, TAN 1511, and TAN 1031A; phorbol esters such as phorbol 12-myristate 13-acetate; cytokines such as interferon, interleukin, and tumor necrosis factor; immunomodulators such as betafectin; and other compounds such as DMPO, Formylated peptides, and opsonified zymosan.

Compounds that deactivate catalase in vivo can be used along with the ultrasound therapy of the present invention. Among the compounds that deactivate catalase in vivo are interleukin-1beta; cumene hydroperoxide; t-butyl hydroperoxide; hydrogen peroxide; toxohormone; and a combination of copper, hydrogen peroxide and ophenanthroline.

Other compounds that can be used in combination with the ultrasound therapy of the present invention include compounds that alter cell membrane permeability so that the cell is more susceptible to lysis or rupture during ultrasound treatment. These compounds also enhance free radical production.

Other compounds that can be used in the present invention to enhance free radical production are those with demonstrated prooxidant activity. Examples include but are not limited to hydrazine derivatives, diamide, t-butylhydroperoxide, hydrogen peroxide, oxygen, and prooxidant drugs such as primaquine. Additionally, compounds traditionally considered to be antioxidants may behave as prooxidants under certain conditions and at certain concentrations. Examples of these compounds are gallic acid, cumene hydroperoxide, endotoxins (e.g., LPS), baiclain, vitamins (K₃, D and E), melatonin, bilirubin, N-(4-hydroxyphenyl)retinamide, beta-hematin, flavone, chalcone, chalconarigenin, naringenin, bleomycin, platinum derivatives (e.g., cisplatin), nitrogen and sulfur mustards, primaquine, manadione, a-tocopherol, β-carotene, Trolox C, estrogen, androgens (e.g., 5-alhpa-DHT), 1,4-naphthoquinone-2-methyl-3-sulfonate, ascorbic acid gallic acid, captopril, enalapril, buthionine, sulfoximine, N-ethylmaleimide, and diazenedicarboxylic acid bis (N,N′-dimethylamide), heme and its degradation products (bile pigments) and heme precursors.

Compounds that exhibit increased thiobarbituric acid reactive substances (TBARS) in the presence of a metal and hydrogen peroxide are known to promote radical production, usually via a Fenton and Haber-Weiss reaction mechanism. These compounds are therefore suitable candidates for use in sonodynamic and photodynamic therapy. More preferably, compounds that exhibit increased thiobarbituric acid reactive substances (TBARS) in the presence of a metal, hydrogen peroxide, and a radical generating source such as an enzymatic source or a radiolytic source are excellent candidates for use as sonodynamic agents, since ultrasound can be substituted as the radical generating source.

The following chelants increase free radical production when exposed to ultrasound and a metal: aminocarboxylates and their salts, derivatives, isomers, polymers, and iron coordination compounds. Addition of a reducing agent such as ascorbic acid, 1,4-naphthoquinone derivatives, 1,4 benzoquinone derivatives, and 1,4-anthraquinone derivatives and/or thiols further increases radical production. This was demonstrated using the following aminocarboxylate chelants:

Ethylenediaminetetraacetic acid
Ethylene glycol-bis(2-aminoethyl)-N,N,N′,N′-
tetraacetic acid
Diaminocyclohexane-N,N,N′,N′-tetraacetic acid
Nitriloacetic acid
N-(2-Hydroxyethyl)ethylenediamine-N,N′,N′-
triacetic acid
Diethylenetriaminepentaacetic acid
Picolinic acid

Examples of other aminocarboxylate chelants are diethylenediamine pentaacetic acid, ethylenediaminedisuccinic acid (EDDS), iminodisuccinate (IDSA), methylglycinediacetic acid (MGDA), glutamate, N,N-bis (carboxymethyl) (GLUDA), diethylenetetraaminepentaacetic acid (DTMPA), ethylenediaminediacetic acid (EDDA), 1,2-bis(3,5-dioxopiperazine-1-yl)propane (ICRF-187), and N,N′-dicarbozamidomethyl-N,N′-dicarboxymethyl-1,2-diaminopropane (ICRF-198). This list is representative of chelants based on the aminocarboxylate structure and is not all inclusive.

Chelants that have available a coordination site that is free or occupied by an easily displaceable ligand such as water are preferred; however this is not a strict requirement for activity.

In general, a 0.5:1 to 10:1 ratio of chelant to metal is preferred (Graf, (1984); Thomas, (1993); Inoue (1987)).

The following chelants increase free radical production when exposed to ultrasound and a metal: hydroxycarboxylate chelants and related compounds including organic alpha and beta hydroxycarboxylic acids, alpha and beta ketocarboxylic acids and salts thereof, their derivative, isomers, metal coordination compounds, and polymers. We demonstrated this using citrate. The chelant should be present in a 0.5:1 to 100:1 ratio of chelant to metal. More preferably a ratio of 0.5:1 to 30:1 (chelant:iron) should be used. Addition of a reducing agent such as ascorbic acid, 1,4-naphthoquinone derivatives, 1,4 benzoquinone derivatives, and 1,4-anthraquinone derivatives and/or thiols further increases radical production.

Chelants that have available a coordination site that is free or occupied by an easily displaceable ligand such as water are preferred; however this is not a strict requirement for activity.

Examples of other compounds are tartaric acid, glucoheptonic acid, glycolic acid, 2-hydroxyacetic acid; 2-hydroxypropanoic acid; 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxypropanoic acid; 2,3-dihydroxypropanoic acid; 2,3,4-trihydroxybutanoic acid; 2,3,4,5-tetrahydroxypentanoic acid; 2,3,4,5,6-pentahydroxyhexanoic acid; 2-hydroxydodecanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid; isocitric acid; agaricic acid; quinic acid; glucuronic acid; glucuronolactone; galacturonic acid; galacturonolactone; uronic acids; uronolactones; dihydroascorbic acid; dihydroxytartaric acid; tropic acid; ribonolactone; gluconolactone; galactonolactone; gulonolactone; mannonolactone; ribonic acid; gluconic acid; citramalic acid; pyruvic acid; hydroxypyruvic acid; hydroxypyruvic acid phosphate; methylpyruvate; ethyl pyruvate; propyl pyruvate; isopropyl pyruvate; phenyl pyruvic acid; methyl phenyl pyruvate; ethyl phenyl pyruvate; propyl phenyl pyruvate; formyl formic acid; methyl formyl formate; ethyl formyl formate; propyl formyl formate; benzoyl formic acid; methyl benzoyl formate; ethyl benzoyl formate; propyl benzoyl formate; 4-hydroxybenzoyl formic acid; 4-hydroxyphenyl pyruvic acid; and 2-hydroxyphenyl pyruvic acid. This list is representative of chelants based on the hydroxycarboxylic acid and ketocarboxylic acid structure but is not all inclusive (Toyokuni, (1993)).

The following chelants increase free radical production when exposed to ultrasound and a metal: adenosine diphosphate (ADP), adenosine triphosphate (ATP) and guanosine triphosphate (GTP). In general, a 0.5:1 to 10:1 ratio of chelant to metal is preferred. Addition of a reducing agent such as ascorbic acid, 1,4-naphthoquinone derivatives, 1,4 benzoquinone derivatives, and 1,4-anthraquinone derivatives and/or thiols further increases radical production. We demonstrated this using ADP.

The following compounds increase free radical production when exposed to ultrasound and a metal: phosphonoformic acid, phosphonoacetic acid, and pyrophosphate. In general, a 0.5:1 to 30:1 ratio of compound to metal is preferred. These compounds can act as chelants and/or reducing agents. We demonstrated the activity of these compounds when phosphonoformic acid was added to an iron/EDTA system and radical production was increased.

Addition of a chelant such as aminocarboxylates, hydroxycarboxylates, or biologically relevant chelants such as ADP, ATP, or GTP further increases radical production. Addition of a reducing agent such as ascorbic acid, 1,4-naphthoquinone derivatives, 1,4 benzoquinone derivatives, and 1,4-anthraquinone derivatives and/or thiols further increases radical production (Lindqvist, (2001)).

The following compounds increase free radical production when exposed to ultrasound and a metal: tetracycline antibiotics and their derivatives, salts, and polymers. These compounds can act as chelants and/or reducing agents. We demonstrated the activity of these compounds when tetracycline was added to iron and radical production was increased. Examples include but are not limited to methacycline, doxycycline, oxytetracycline, demeclocyline, meclocycline, chlortetracycline, bromotetracycline, daunomycin, dihydrodaunomycin, adriamycin, steffimycin, steffimycin B, 10-dihydrosteffimycin, 10-dihydrosteffimycin B, 13213 RP, tetracycline ref. 7680, baumycin A2, baumycin A1, baumycin B1, baumycin B2, antibiotic MA 144S1, rhodomycin antibiotic complex, musettamycin, antibiotic MA 144L1, aclacinomycin B, antibiotic MA 144 Y, aclacinomycin A, antibiotic MA 144G1, antibiotic MA 144M1, antibiotic MA 144N1, rhodirubin B, antibiotic MA 144U1, antibiotic MA 144G2, rhodirubin A, antibiotic MA 144M2, marcellomycin, serirubicin, oxytetracycline, demeclocycline and minocycline.

Addition of a chelant such as aminocarboxylates, hydroxycarboxylates, or biologically relevant chelants such as ADP, ATP, or GTP further increases radical production Addition of a reducing agent such as ascorbic acid, 1,4-naphthoquinone derivatives, 1,4 benzoquinone derivatives, and 1,4-anthraquinone derivatives and/or thiols further increases radical production (Quinlan, (1998)).

The following compounds increase free radical production when exposed to ultrasound and a metal: hydroxy-1,4-naphthoquinones, their derivatives, isomers, metal coordination compounds, salts, and polymers. These compounds can act as chelants and/or reducing agents. We demonstrated their effectiveness using the following compounds:

10 uM 5-hydroxy-1,4-naphthoquinone
(juglone)
15 uM 2-hydroxy-3-(3-methyl-2-butenyl)-
1,4-naphthoquinone (lapachol)
71 uM 5-hydroxy-2-methyl-1,4-
naphthoquinone (plumbagin)
106 uM 5,8 dihydroxy -1,4-naphthoquinone

Addition of a chelant such as aminocarboxylates, hydroxycarboxylates, or biologically relevant chelants such as ADP, ATP, or GTP further increases radical production. Addition of a reducing agent such as ascorbic acid, 1,4 benzoquinone derivatives, and 1,4-anthraquinone derivatives and/or thiols further increases radical production.

Other examples of hydroxylated 1,4-naphthoquinones include the following compounds and their derivatives: 1,4-naphthalenedione, 2,3-dihydroxy; 1,4-naphthalenedione, 2,5,8-trihydroxy; 1,4-naphthalenedione, 2-hydroxy; 1,4-naphthalenedione, 2-hydroxy-3-(3-methylbutyl); 1,4-naphthalenedione, 2-hydroxy-3-methyl; 1,4-naphthalenedione, 5,8-dihydroxy-2-methyl; alkannin; alkannin dimethylacrylate; aristolindiquinone, chleone A, droserone; isodiospyrin; naphthazarin; tricrozarin A, actinorhodine, euclein, and atovaquone. This list is representative of hydroxy-1,4-naphthoquinones and is not all inclusive.

The following compounds increase free radical production when exposed to ultrasound and a metal: hydroxylated 1,4-benzoquinones, their derivatives, isomers, metal coordination compounds, salts, and polymers. These compounds can act as chelants and/or reducing agents. We demonstrated their effectiveness using the following compound:

Tetrahydroxy 1,4-benzoquinone

Addition of a chelant such as aminocarboxylates, hydroxycarboxylates, or biologically relevant chelants such as ADP, ATP, or GTP further increases radical production. Addition of a reducing agent such as ascorbic acid, 1,4-naphthoquinone derivatives, and 1,4-anthraquinone derivatives and/or thiols further increases radical production.

Embelin, methylembelin, and rapanone are examples of other hydroxylated 1,4-benzoquinones.

The following compounds increase free radical production when exposed to ultrasound and a metal: hydroxylated anthraquinones, their derivatives, isomers, metal coordination compounds, salts, and polymers. These compounds can act as chelants and/or reducing agents.

Addition of a chelant such as aminocarboxylates, hydroxycarboxylates, or biologically relevant chelants such as ADP, ATP, or GTP further increases radical production. Addition of a reducing agent such as ascorbic acid, 1,4-naphthoquinone derivatives, 1,4 benzoquinone derivatives, and/or thiols further increases radical production.

Examples of hydroxylated anthraquinones include but are not limited to the following compounds and their derivatives: alizarin, aloe-emodin, anthragallol, aurantio-obtusin, barbaloin, cascaroside A, cassiamin C, 7-chloroemodin, chrysazin, chryso-obtusin, chrysophanic acid 9-anthrone, digiferrugineol, 1,4-dihydroxy-2-methylanthraquinone, frangulin A, frangulin B, lucidin, morindone, norobtusifolin, obtusifolin, physcion, pseudopurpurin, purpurin, danthron, and rubiadin. Prodrugs such as diacerein that are converted to hydroxylated anthraquinones in the body are also relevant (Kagedal, (1999); Lee, (2001); Lee, et al. (2001); Gutteridge, (1986); Muller, (1993)).

Flavonoids such as kaempferol, quercetin, and myricetin and sesquiterpenes such as gossypol and feralin are reducing agents and/or chelants that increase free radical production when exposed to ultrasound and a metal. Addition of a chelant such as aminocarboxylates, hydroxycarboxylates, or biologically relevant chelants such as ADP, ATP, or GTP further increases radical production. Other examples of flavonoids include, but are not limited to acacetin, apigenin, biochanin-A, daidzein, equol, flavanone, flavone, formononetin, genistin, glabranin, liquiritigenin, luteolin, miroestrol, naringenin, naringin, phaseollin, phloretin, prunetin, robinin, and sophoricoside. Derivatives, polymers, and glycosylated forms of these compounds are also relevant. B-dihydroxy and B-trihydroxy flavonoids are preferred (Canada, (1990); Laughton, (1989)).

The following compounds increase free radical production when exposed to ultrasound and a metal: anti-tumor antibiotic quinoid agents such as benzoquinones, mitomycin, streptonigrins, actinomycins, anthracyclines, and substituted anthraquinones. These compounds can act as chelants and/or reducing agents.

Addition of a chelant such as aminocarboxylates, hydroxycarboxylates, or biologically relevant chelants such as ADP, ATP, or GTP further increases radical production. Addition of a reducing agent such as ascorbic acid or thiols further increases radical production (Gutteridge, (1985); Gutteridge, et al. (1984); Morier-Teissier, et al. (1990)).

The following compounds increase free radical production when exposed to ultrasound and a metal: ascorbic acid, its derivatives, salts and polymers act as ultrasound enhanced reducing agents and/or chelants. Addition of a chelant such as aminocarboxylates, hydroxycarboxylates, or biologically relevant chelants such as ADP, ATP, or GTP further increases radical production (Schneider, (1988); Dognin, (1975)).

Thiol compounds, their derivatives, and polymers increase free radical production when exposed to ultrasound and a metal. We demonstrated their effectiveness using cysteine as an example of a biological thiol and pennicillamine as an example of a thiol drug. Biological thiols and thiol drugs are preferred. Examples of biological thiols include, but are not limited to cysteinylglycine, cysteamine, thioglycollate and glutathione. Other thiol containing drugs include but are not limited to Captopril, Pyritinol (pyridoxine disulfide), Thiopronine, Piroxicam, Thiamazole, 5-Thiopyridoxine, Gold sodium thiomalate, and bucillamine. In addition, drugs classified as penicillins, cephalosporins, and piroxicam may undergo hydrolytic breakdown in vivo to form thiols; therefore, they are thiol prodrugs. Addition of a chelant such as aminocarboxylates, hydroxycarboxylates, or biologically relevant chelants such as ADP, ATP, or GTP further increases radical production. Addition of a reducing agent such as ascorbic acid, 1,4-naphthoquinone derivatives, 1,4 benzoquinone derivatives, and/or 1,4-anthraquinone derivatives.

A comprehensive list of thiol compounds include 1-(mercaptomethyl)-7,7-dimethylbicyclo[2.2.1]heptan-2-one; 1,2,3-benzotriazine-4(3H)-thione; 1,2-benzisothiazole-3(2H)-thione-1,1-dioxide;1,2-dihydro-3H-1,2,4-triazole-3-thione; 1,2-dihydro-3H-1,2,4-triazole-3-thione and derivatives; 1,2-dihydro-4,5-dimethyl-2H-imidazole-2-thione; 1,3-dihydro-1-methyl-2H-imidazole-2-thione; 1,3-dihydro-2H-naphth[2,3-d]imidazole-2-thione; 1,3-dihydro-4,5-diphenyl-2H-imidazole-2-thione; 1,4-benzoxazepine-5(4H)-thione; 1,4-dihydro-5H-tetrazole -5-thione and derivatives; 1,5-dihydro-4H-pyrazolo[3,4-d]pyrimidine-4-thione; 1,5-dihydro-6H-imidazo[4,5-c]pyridazine-6-thione; 1,7-dihydro-6H-purine-6-thione; 1-adamantanethiol; 2(1H)-benzimidazolinethione; 2,4-diamino-6-mercapto-1,3,5-triazine; 2,4-dimethylbenzenethiol; 2,5-dimethylbenzenethiol; 2,6-dimethylbenzenethiol; 2-adamantanethiol; 2-amino-1,7-dihydro-6H-purine-6-thione; 2H-1,4-benzothiazine-3(4H)-thione; 2-imidazolidinethione; 2-Isopropyl-3-methylbenzenethiol; 2-isopropyl-4-methylbenzenethiol; 2-isopropyl-5-methylbenzenethiol; 2-mercapto-4H-1-benzopyran-4-thione; 2-mercapto-5-methyl-1,3,4-thiadiazole; 2-mercapto-5-nitrobenzimidazole; 2-mercaptothiazoline; 2-methyl-1-propenethiol; 2-methylene-1,3-propanedithiol; 2-propene-1-thiol; 3,4-dihydro-4,4,6-trimethyl-1-(4-phenyl-2-thiazolyl)-2(1H)-pyrimidinethione; 3,4-dihydro-4,4,6-trimethyl-2(1H)-pyrimidinethione; 3-amino-5-mercapto-1H-1,2,4-triazole; 3-bromo-1-adamantanethiol; 3-mercapto-5-methyl-1,2,4-triazole and derivatives; 3-mercaptocyclohexanone and derivatives; 3-quinuclidinethiol; 3-thio-9,10-secocholesta-5,7,10(19)-triene; 4-amino-2,4-dihydro-5-phenyl-3H-1,2,4-triazole-3-thione; 4-amino-3-hydrazino-5-mercapto-1,2,4-triazole; 4-benzocyclobutenethiol; 4-biphenylthiol; 4-Isopropyl-2-methylbenzenethiol; 5,6-dichloro -2-mercapto-1H-indole; 5′-amino-2′,3,3′,4-tetrahydro-4,4,6-trimethyl-2,2′-dithioxo[1(2H),4′-bipyrimidin]-6′(1′H)-one; 5-isopropyl-2-methylbenzenethiol; 5-mercapto-3-methyl-1,2,4-thiadiazole; 6-amino-2-mercaptopurine; 6-thioinosine; 7-(mercaptomethyl)-1,7-dimethylbicyclo[2.2.1]heptan-2-one; 7-mercapto-3H-1,2,3-triazolo[4,5-d]pyrimidine; Azothiopyrine; benzo[c]thiophene-1(3H)-thione; bis(1-methylethyl)carbamothioic acid S-(2,3,3-trichloro-2-propenyl) ester; Caesium [2,6-bis(2,4,6-triisopropylphenyl)phenyl]thiolate; (3β)-cholest-5-ene-3-thiol; Cyclohexanethione; Lithium [2,6-bis(2,4,6-triisopropylphenyl)phenyl]thiolate; naphtho[1,2-d]thiazole -2(1H)-thione; naphtho[2,1-d]thiazole-2(3H)-thione; phenylmethanethiol; Potassium [2,6-bis(2,4,6-triisopropylphenyl)phenyl]thiolate; Rubidium [2,6-bis(2,4,6-triisopropylphenyl)phenyl]thiolate; Sodium [2,6-bis(2,4,6-triisopropylphenyl)phenyl]thiolate (Diez, (2001)).

Sodium sulfide and sodium sulfite are reducing agents that increase free radical production when exposed to ultrasound and a metal. We demonstrated this using sodium sulfite. Addition of a chelant such as aminocarboxylates, hydroxycarboxylates, or biologically relevant chelants such as ADP, ATP, or GTP further increases radical production (Cassanelli, (2001)). By screening compounds using the TBARS assay in combination with ultrasound exposure, one skilled in the art can readily identify compounds that are particularly active during ultrasound exposure.

More preferably, compounds that exhibit increased thiobarbituric acid reactive substances (TBARS) in the presence of a metal, hydrogen peroxide, and a radical generating source such as an enzymatic source or a radiolytic source, are excellent compounds for use as sonodynamic agents activated by ultrasound. Thus, using the TBARS assay with ultrasound exposure as the radical generating source, one skilled in the art can readily identify useful compounds. The TBARS assay can be used in aqueous, lipid, and biological systems.

Other compounds that can be used in the present invention are those that exhibit iron release from biological compounds containing iron, such as ferritin, hemoglobin, transferrin, etc., in the presence of ultrasound. For example, anthraquinones are known to release iron from ferritin during exposure to a free radical generating source such as a radiolytic or enzymatic source. By screening quinone compounds using an assay for the release of iron from ferritin with ultrasound exposure as the free radical generating source, it is possible to identify suitable quinone sonondynamic agents.

Copper and iron are the best metals for enhancing the Fenton and Haber-Weiss activity in the body, and thus are the preferred metals for use in the present invention. Platinum and chromium are also preferred metals.

The compounds described above for use as sonodynamic agents can be modified to increase their solubility. Glycolysed or cyclodextrin modified compounds are some examples.

In one embodiment, high levels of ascorbic acid are administered to a diseased body, followed by administration of liposomally or polymerically encapsulated Fe(II). Ultrasound is used to rupture the liposome or polymer capsule to release iron at the target tissue. Ascorbic acid acts as the reductant. Alternatively, ascorbic acid can be encapsulated alone or as part of the iron capsule and administered along with the iron.

Another embodiment is treatment with EDTA, either systemically or encapsulated in a bead. The bead is ruptured at the treatment site with ultrasound or other exogenous energy sufficient to rupture the material of which the capsule is made. Treatment is guided with ultrasound imaging.

Ultrasound mobilizes iron either reductively from biological storage or by degradation of heme compounds. Alternatively, iron is added to the EDTA prior to treatment or delivered separately. The iron, regardless of its source, chelates with EDTA and remains soluble and able to generate free radicals and reactive oxygen species. The addition of ascorbic acid or thiols or sulfate or hydroxylated 1,4-naphthoquinones (either systemically or encapsulated) enhances the production of free radical and reactive oxygen species.

Quinones are well suited reductants in this invention, since they are only active in their semiquinone form which can be generated by the application of ultrasound. The source of the quinone compounds can be azo dyes, which are treated by ultrasound to form quinones. These azo dyes can be thought of as prodrugs for quinone compounds under the influence of ultrasound.

For purposes of the present invention, an “activator” means at least one of a transition metal such as iron, a reductant, or a chelant, in any combination. Thus, one could use a transition metal, a reductant, or a chelant alone, or a transition metal plus a reductant or a chelant, or a combination of a transition metal, a reductant, and a chelant.

The present invention also provides a method for preventing development or metastasis of cancer by delivering a combination of a sonodynamic or photodynamic agent and at least one activator which is a transition metal, a reductant, or a chelant to precancerous or cancerous cells to affected tissues or organs of an animal, and then exposing those tissues or organs to irradiation which results in destruction of the cells. For purposes of the present invention, irradiation refers to delivering light or sound waves, or alpha, beta, or gamma emmissions. This enhanced form of sonodynamic therapy or photodynamic therapy can be used in combination with conventional therapeutic regiments including radiation therapy, hormonal therapy, or one or more chemotherapeutic agents.

In another embodiment of the present invention, diseases or conditions which can be treated by destroying tissue, e.g., cardiovascular disease, are treated by administering to the site a combination of a photodynamic agent and/or sonodynamic agent with at least one activator which is a transition metal, a reductant, or a chelant and exposing the tissue to irradiation.

In another aspect of the present invention, infectious diseases are treated by administering a sonodynamic and/or photodynamic compound along with an activator to enhance the formation of free radicals to a patient suffering from an infectious disease in order to destroy the microorganisms causing the disease.

The present invention can be used to enhance the sterilizing effect of irradiation such as light, ultrasound, microwave, etc., to destroy unwanted microorganisms by administering to the desired site a combination of a photodynamic agent and/or sonodynamic agent with at least one activator which is a transition metal, a reductant, or a chelant and exposing the site to irradiation to destroy the pathogens.

The present invention can also be used to arrest bleeding by delivering a combination of a photodynamic agent or sonodynamic agent with an activator to the site of bleeding and exposing the site to irradiation.

According to the present invention, at least one sonodynamic and/or photodynamic agent and an activator are combined prior to treating a site, such as a patient, surface, or reaction medium, with light or sound energy. For treating diseases and conditions, at least one sonodynamic and/or photodynamic agent, in combination with at least one activator, are administered either together or separately as an injection or infusion, or applied directly, to a site. The site is then subjected to the appropriate irradiation, at with which time free radicals are formed which are capable of destroying the tissue or pathogen intended to be destroyed.

### BRIEF DESCRIPTION OF THE DRAWINGS

FIG. 1 illustrates the use of ultrasound to convert methyl orange, o- and p-methyl red, and azobenzene to a quinone.

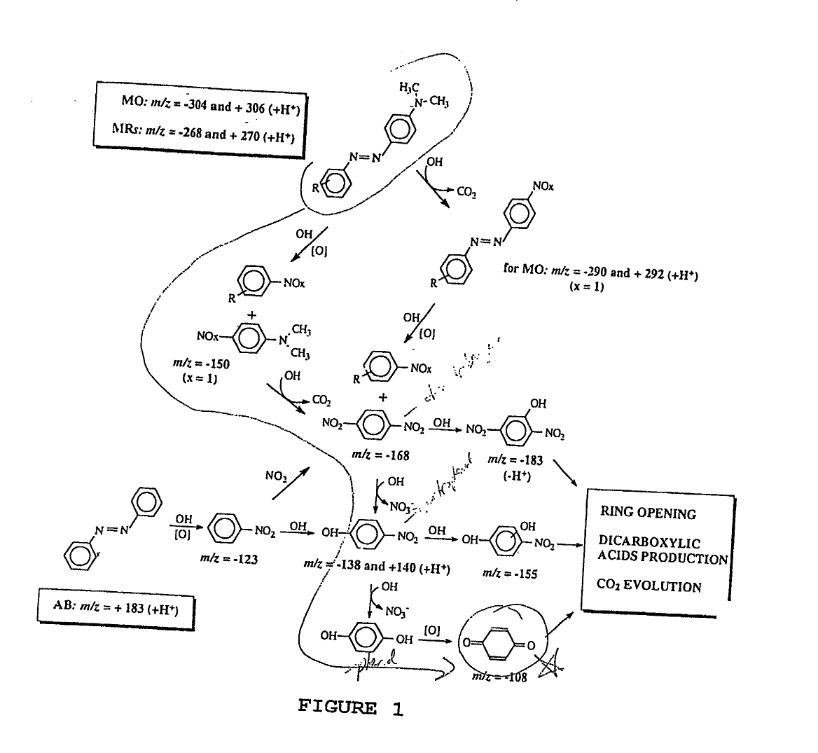

### DETAILED DESCRIPTION OF THE INVENTION

According to the present invention, production of free radicals is enhanced using the combination of at least one sonodynamic agent and/or at least one photodynamic agent in combination with at least one activator. The activator is any combination of a transition metal, a reductant or a chelant. This combination is then treated at the desired site, e.g., reaction medium, with the appropriate light and/or sonic energy to generate free radicals. In another embodiment of the present invention, a human or animal body is treated by sonodynamic and/or photodynamic therapy wherein a photodynamic and/or sonodynamic agent plus at least one activator is administered to said body and the body is exposed to light rays and/or ultrasound to achieve a cytopathogenic effect at a site therein. It has been found that combining at least one activator with a photodynamic and/or sonodynamic agent results in greatly increased rate of production of free radicals, thus greatly enhancing the effects of the photodynamic or sonodynamic therapy.

In another aspect of the present invention, the photodynamic or sonodynamic compound contains a reporter moiety which is detectable by an in vivo diagnostic imaging modality, and optionally a vector moiety which modifies the biodistribution of the photodynamic or sonodynamic compound, e.g., by prolonging the blood residence time of the compound or by actively targeting the compound to particular body sites such as disease sites or other proposed sites for PDT or SDT. According to this aspect of the invention, a human or animal body can be treated by photodynamic or sonodynamic therapy wherein a photodynamic or sonodynamic compound which includes a reporter moiety is administered to the body in conjunction with an activator, the body is exposed to light or ultrasound to achieve a cytopathogenic effect at a site therein. In this way, an image of the body to which the photodynamic or sonodynamic compound is distributed makes it possible to locate sites for treatment by light or ultrasound, or to follow the progress of the therapy at a site within the body. Any suitable imaging methods may be used, including X-ray, MRI, ultrasound, light imaging, scintigraphy, in vivo microscopy, such as confocal, photoacoustic imaging, and acousto-optical imaging and visual observation and photographic imaging, magnetotomography, positron emission tomography or electrical impedance tomography.

The choice of reporter moiety used depends on the choice of imaging modality. For X-ray imaging, the reporter is preferably a heavy atom (atomic number greater than 37), a chelated heavy metal ion or complex ion, or a particular substance such as a heavy metal compound, an insoluble iodinated organic compound, or a vesicle enclosing an iodinated organic compound or a heavy metal compound.

For MRI, the reporter is preferably a paramagnetic, superparamagnetic, ferromagnetic, or ferrimagnetic material such as a chelated transition metal or lanthamide ion (such as Gd, Dy, Mn, or Fe), or a superparamagnetic metal oxide particle.

For ultrasound imaging, in which case the imaging and therapy may be effected by the same or similar apparatus, the reported is preferably a particular substance bound to the rest of the photodynamic or sonodynamic compound, such as a vesicle (liposome, micelle, or microballoon) enclosing an echogenic contrast agent such as a gas or a gas precursor (a material which is gaseous at 37 Celcius), or a mixture thereof. Particularly useful echogenic materials are perfluoroalkanes such as perfluoropentane and perfluorobutane.

For scintigraphy, the reporter is generally a covalently bound non-metal radionuclide such as an iodine isotope.

For light imaging, the reporter is a chromophore i.e., a compound which absorbs light at 300-1300 nm, preferably 600 to 1300 nm, and includes fluorophores and phosphorescent materials, and/or light scatterers such as particulates with or without associated chromophores. Reporters for magnetotomography include materials useful as magnetic resonance reporters, particular chelated lanthamides or superparamagnetic metal oxides.

A more detailed list of reporters that can be used in the present invention is given in Alfheim et al., PCT application WO 98/52609, the entire contents of which are hereby incorporated by reference.

For electrical impedance tomography, the reporter is preferably a polyelectrolyte.

Imaging may be affected in a conventional fashion and using conventional imaging apparatus for the selected imaging modality. The reporter-containing photodynamic or sonodynamic compound plus metal is administered in a contrast-enhancing dose, e.g., a dose conventional for the selected imaging procedure, or at lower than conventional dose where the agent is administered near the target site for SDT or PDT or where it is actively targeted to the target site by a vector moiety.

The photodynamic or sonodynamic compound may optionally include a vector moiety which modifies the biodistribution of the compound. Example of suitable vectors include antibodies, antibody fragments, proteins and oligopeptides which have affinity for cell surface receptors, especially receptors associated with surfaces of diseased or rapidly proliferating cells, and peptidic and non-peptidic drugs which are preferentially taken up by diseased or rapidly proliferating cells.

Definitions

Unless indicated otherwise, the following definitions obtain for the present invention. All percentages are by weight unless otherwise indicated.

Ultrasound comprises sound waves that occur at a frequency above the audible frequency of the human ear (16 kHz). Ultrasound is generally associated with frequencies of about 20 kHz to about 500 MHZ.

Cavitation is the formation of vapor bubbles during the negative pressure cycle of ultrasound waves. The bubbles can collapse, resulting in localized high temperatures and pressures. Free radicals, such as the hydroxyl radical hydrogen radical, singlet oxygen, and solvated electrons are typically generated form bubble collapse in aqueous media.

Medical imagining involves the use of electromagnetic radiation to produce images of internal structures of the human body for purposes of accurate diagnosis. Four imaging modalities are most commonly used in medical practice for diagnosis and therapy: ultrasound, MRI, X-rays, and nuclear medicine.

Contrast agents are pharmaceutical agents that are used in many medical imaging examinations to aid in visualizing tumors, blood vessels, and other structures. For example, gas filled microspheres are used as a contrast agent for ultrasound imaging. Paramagnetic compounds can be used as MRI contrast agents.

Irradiation for purposes of the present invention refers to any type of irradiation which is biologically compatible. This includes visible light, infrared light, ultraviolet light, ultrasound, microwaves, radio waves, laser light, magnetic files, or X-rays. Irradiation can be applied singly as a continuous wave or can be pulsed. Each type of irradiation can be applied in combination and/or sequentially with one or more additional types of irradiation.

Photodynamic therapy involves the combined use of photosensitizable compounds plus an appropriate light source to generate a cytotoxic effect. In the present invention, a metal is present to enhance this effect. The photosensitizable compound is capable of absorbing or interacting with at least one specific wavelength of light. This wavelength defines the type of irradiation used in photodynamic therapy. Generally, a visible wavelength of light provided by laser is used.

Sonodynamic therapy involves the combined use of sonosensitizable compounds plus an appropriate ultrasound source to generate a cytotoxic effect. The sonosensitizable compound is capable of absorbing or interacting with the ultrasound irradiation. For purposes of the present invention, the sonosensitizable compound is used in combination with a metal. Ultrasound within a frequency range of about 1 kHz to about 100 MHZ is generally used, with intensities of about 0.1 W/cm ² to about 10,000 W/cm². High intensity focused ultrasound (HIFU) can deliver intensities of up to 10,000 W/cm², with values typically in the range of 500-2,000 W/cm². Ultrasound irradiation is generally applied from about 0.5 sec to about five hours, depending on the frequency, intensity, material treated, etc., as is well appreciated by one skilled in the art. The ultrasound can be pulsed, second harmonic, or continuous wave. Custom built systems can be used, or commercial diagnostic or therapeutic devices can be used in practicing the present invention. The particular type of apparatus used is not critical.

Ligands are negatively charged chemicals that combine with a positively charged metal. Monoatomic examples are F—, Cl—, etc. Polyatomic examples are NH₃, CNS—, H₂OH, etc.

Ligands are classified by the number of coordination sites available:

1 site = monodentate
2 sites = bidentate
3 sites = tridentate
4 sites = tetradentate
6 sites = hexadentate

Monodentate ligands are Cl—, NH₃, CN—, and F—. Examples of bidentate ligands are 1,10-phenanthroline and ethylene diamine.

Chelates are complex ions that involve ligands with two or more bonding sites.

Chelants or chelating agents are ligands with two or more bonding sites.

Diagnostic or therapeutic ultrasound elements can be based on any method for focusing ultrasound, including geometric, annular, or phase array, and the probe can include both therapeutic and imaging capabilities. Focused or direct ultrasound refers to the application of ultrasound energy to a particular region of the body, such that the energy is concentrated to a selected area or target zone. Devices that are designed for administering ultrasound hyperthermia are also suitable, as are ultrasound devices used in surgery, such as high intensity focused ultrasound devices.

Transition metals which are preferred for use in the present invention are those that can produce and/or react with molecular oxygen or molecular oxygen derived reactive species, such as hydrogen peroxide and superoxide. This interaction is preferably via a Fenton and/or Haber-Weiss mechanisms, or mechanisms related to the Fenton and Haber-Weiss reactions, such as radical-driven Fenton reactions. Iron, copper, manganese, molybdenum, cobalt, vanadium, chromium, nickel and zinc are of particular pharmacological importance. The (I), (II), (III), (IV), and/or (V) oxidation states or higher, and combinations thereof, depending upon the choice of metal(s), may be used. Water-soluble or lipid-soluble forms of the transition metals can be used. The metal can be administered in the form of free metal, or chelated or bound entities. The chelators may be free molecular entities or prosthetic groups in larger molecules (e.g., porphyrin in hemoproteins).

Ferritin is a preferred vehicle for iron delivery in vivo. This protein contains up to 4500 atoms of Fe(III) which can be released as Fe(II) by the application of ultrasound. Furthermore, ferritin can be modified to include surface moieties which enhance the release of iron or Fenton reactions. For example, reducing agents which are only active upon exposure to ultrasound will both aid the release of iron from ferritin but will also engage in radical driven Fenton reactions. Non-enzymatically loaded ferritin may be used, which has shown a greater ability to release iron. While ferritin is the preferred biological source of iron, other biological sources of iron can be used in the present invention.

Other biological sources of iron or other metals such as transferrin, lactoferrin, conalbumin, ovotransferrin, cytochrome C, heme compounds, myoglobin, porphyrin and porphyrin containing macromolecules, and metal containing co-factors can be utilized. Synthetic versions, modifications or complexes of these compounds are also suitable.

Particulate forms of transition metals or combinations of metals in particulate form can be used.

The metal chelator can be chosen to enhance the Fenton chemistry by maintaining the transition metal in a redox-active form and/or by lowering the redox potential of the metal. This enables the transition metal to act as a prooxidant. A classic example is EDTA, which chelates iron and lowers the redox potential of Fe(III)/Fe(II) by 0.65V. This greatly favors the reaction of iron with hydrogen peroxide to form the toxic hydroxyl radical species. Other such chelators typically used with iron include nitrilotriacetic acid (NTA), penicillamine (PCM), and triethylene tetramine (TTM). Additional chelants can also be used, including hydroxyethyleniminodiacetate (HEIDA), gallate (GAL), hexaketocyclohexane, tetrahydroxy-1,4-quinone, gallic acid, rhodizonic acid, dipicolinic acid, alizarin, ascorbic acid, and picolinic acids. Other examples are given in U.S. Pat. Nos. 6,160,194 and 5,741,427, the entire contents of which are hereby incorporated by reference. Flavonoids can also be used as metal ion chelators which reduce the redox potential of metal ions.

The choice of reductant can be guided by its redox potential, such that the reduction of the transition metal back to the active form after it has participated in the radical producing reaction is thermodynamically favorable. For example, ascorbic acid has a standard reduction potential of −0.127V, and is therefore able to reduce Fe(III) to Fe(II), where the Fe(III)/Fe(II) standard reduction potential is 0.77V. The Fe(II) form is then able to react with species such as hydrogen peroxide, with the production of radical species such as hydroxyl radical ion.

Reducing agents are often metal chelators. For example, oxalate can chelate iron and reduce it from Fe(III) to Fe(II).

In preferred modes utilizing ultrasound, such as sonodynamic therapy, the preferred reducing agent is a species which is activated by ultrasound. Such species readily becomes a radical upon exposure to ultrasound, and exhibits no cytotoxic behavior in the absence of ultrasound. Compounds containing a quinone structure are preferred compounds, and the most preferred quinones are hydroxylated 1,4-naphthoquinones, which are activated by ultrasound and remain inactive without ultrasound. Upon activation by ultrasound they form a semiquinone radical which can then reduce metals. 1-4 benzoquinone and 1-2 benzoquinone, which are also preferred quinones, are the simplest quinones which can be used. Higher molecular weight compounds which contain 1-4 benzoquinone or 1-2 benzoquinone moieties can be used. Such structures include napthoquinones, anthraquinones, and mitomycins. Examples include, but are not limited to, acamelin, alizarin, alkannin, arisianone, arstolindiquinone, barbaloin, cassiamin, cypripedin, 2,6-dimethoxybenzoquinone, diospyrin, embelin, echinone, lapachone, juglone, isodiospyrin, hypericin, lawsone, primin, ubiquinones, rapanone, ramentaceone, sennoside, vitamin K, coenzyme Q, and anthracycline antibiotics.

Additional examples of quinones, both hydroxylated and non-hydroxylated, include, but are not limited to, (p-benzoquinone)bis(triphenylphosphine)palladium; 1,2-naphthalenedione and amino, bromo, butyl, chloro, ethyl, ethynyl, fluoro, hydro, hydroxy, iodo, isopropyl, mercapto, methyl, methoxy, nitro, phenyl, phenylthio derivatives; 1,2-phenanthrenedione and hydroxy, derivatives; 1,4-anthracenedione and derivatives; 1,4-bis[2-(diethylamino)ethoxy]anthraquinone; 1,4-naphthalenedione and amino, bromo, butyl, chloro, ethyl, ethynyl, fluoro, hydro, hydroxy, iodo, isopropyl, mercapto, methyl, methoxy, nitro, phenyl, phenylthio derivatives; 1,4-phenanthrenedione and derivatives; 1,8-diphenyl-1,7-octadiyne-3,6-dione; 11,12,13-Trinor-4-amorphene-3,8-dione; 2-(3-methyl-2-butenyl)-1,4-benzenediol;2-(beta-D-glucopyranosyloxy)-1-hydroxy-9,10-anthracenedione; 2,5-cyclohexadiene-1,4-dione and amine, bromo, carboxyl, chloro, ethoxy, ethyl, fluoro, hydroxyl, methoxy, methyl, nitorso, and phenyl derivatives; 2,5-dichloro-3,6-bis(p-nitroanilino)-p-benzoquinone; 2,6-dimethylbenzoquinone; 2-demethylmultiorthoquinone; 2-ethoxy -2a,3,4,5,5a,6,10b,10c-octahydro-5-hydroxy-8-methoxy-5a-methyl -2H-anthra[9,1-bc]furan-7,10-dione; 2-Geranylemodin 005; 2-Hydroxygarveatin B; 2-methoxy-5-[(1-phenyl-1H-tetrazol-5-yl)thio]-p-benzoquinone; 2-methylconospermone; 2-tetradecyl -1,4-benzenediol; 3,4-dihydro-6(2H)-quinolinone; 3,4-phenanthrenedione and derivatives; 3,5-cyclohexadiene-1,2-dione; 3-[(6-deoxy-alpha-L-mannopyranosyl)oxy]-1,8-dihydroxy-6-methyl-9,10-Anthracenedione; 3-tert-butyl-5,8-dimethyl-1,10-anthraquinone; 4,5-dichloro-3,6-dioxo-1,4-cyclohexadiene-1,2-dicarbonitrile; 4,5-phenanthrenedione and derivatives; 5,10-dihydro-5,10-dioxo-naphtho[2,3-b]-1,4-dithiin-2,3-dicarbonitrile; 5,12-naphthacenedione; 5,6-dihydroxy-naphtho[2,3-f]quinoline-7,12-dione; 5-Methylaltersolanol A;6,13-pentacenedione; 6,15-dihydro-5,9,14,18-anthrazinetetrone; 6,6′-biembelin; 6-[2-(4,9-dihydro-8-hydroxy-5,7-dimethoxy-4,9-dioxonaphtho[2,3-b]furan-2-yl-1H-2-benzopyran-3-carboxylic acid;7-beta-D-glucopyranosyl-9,10-dihydro-3,5,6,8-tetrahydroxy-1-methyl-9,10-dioxo-2-Anthracenecarboxylic acid; 9,10-anthracenedione and amine, azido, benzoyl, bromo, chloro, ethyl, ethenyl, fluoro, hydroxyl, methoxy, methyl, nitroso, and phenyl derivatives; 9,10-phenanthrenedione and amino, bromo, chloro, fluoro, hydroxy, methyl, and nitro derivatives; Acequinocyl; Aclacinomycin A; Actinorhodine; Alizarin Cyanin Green F; Alkannin; Aloesaponol I; Aloetic acid; Altersolanol G; Ametantrone; Aminoanthraquinones and carboxylic acid derivatives; Anthraflavone; Anthrimide; Antibiotic BE 69785A; Antibiotic JTNC; Antibiotic Q 6916Z; Asterriquinone; Atovaquone; Aurantiogliocladin; Austrocortilutein; Austrocortirubin; Averantin; Averythrin; Azanzone A; Benz[a]anthracene-7,12-dione; Benzoquinoniom Cl; Betulachrysoquinone; Bis-(4-amino-1-anthraquinonyl)amine; Bis(phenanthrenequinone)bis(pyridine)nickel; Bostrycin; Bostrycoidin; Buparvaquone; C.I. Vat Yellow; C.I. Violet 43; Canaliculatin; Carboquone; Carubicin; Cassumunaquinone 1; Conospermone; Cordeauxione; Cordiachrome A, B, and C; Cycloleucomelone; Daunorubicin; Decylplastoquinone; Decylubiquinone; Dermoquinone; Diacerein; Diaziquone; Dibenz[a,h]anthracene-7,14-dione; Didyronic acid; Dihydodioxoanthracenesulfonic acid derivatives; Dihydrodioxoanthracenedicarboxylic acid and derivatives; Doxorubicin; Echinochrome A; Epirubicin; Frangulin A and B; Fredericamycin A; Frenolicin; Fusarubin; Geldanamycin; Gossyrubilone; Granaticin; Granatomycin D; Herbimycin A; Idarubicin; Ilimaquinone; Isocordeauxione; Isofusarubin; Javanicin; Juglomycin F; Kermesic acid; Laccaic acid A, B, C, and D; Lagopodin A; Lapinone; Latinone; Leucoquinizarin; Mansonone A,C, and G; Menaquinone 4, 6, and 7; Menatetrenone; Menoctone; Menogaril; Miltirone; Mimocin; Mimosamycin; Mitomycin A, B, and C; Mitoxantrone; Mollisin; Morindin; Murayaquinone; Murrapanine; Mycenone; Mycochrysone; N-(4-amino-3-methyl-1-anthraquinonyl)-benzamide; N-(4-amino-9,10-dihydro-3-methoxy-9,10-dioxo-1-anthracenyl)-4-methyl-benzenesulfonamide; N-(4-amino-9,10-dihydro-9,10-dioxo-1-anthracenyl)-benzamide; N-(4-chloro-9,10-dihydro-9,10-dioxo-1-anthracenyl)-benzamide; N-(5-amino-9,10-dihydro-9,10-dioxo-1-anthracenyl)-benzamide; N,N′-(9,10-dihydro-9,10-dioxo-1,4-anthracenediyl)bis-benzamide; Naphthoherniarin; Naphthomevalin; Naphthyridinomycin A; Nogalamycin; Norjavanicin; Novarubin; Oncocalyxone A; Oosporein; Paeciloquinone A; Parvaquone; Perezone; Phenicin; Piloquinone; Pirarubicin; Pleurotin(e); Porfiromycin; Resistomycin; Rhacodione B; Rhodocomatulin; Rhodomycin A and B; Rhodoquinone; Ruberytheric acid; Rubianin; Seratrodast; Sodium β-naphthoquinone-4-sulfonate; Sodium alizarinesulfonate; Solaniol; Spiranthoquinone; Streptonigrin; Sudan blue GA; Tabebuin; Tectoleafquinone; Triaziquone; Triptone; Ubiquinone 30 and 50; Versiconol; Vitamin K₁; Xanthoviridicatin D; Zorubicin.

Bipyridyl herbicides, such as paraquat and diquat, and compounds containing the bipyridyl structure, are also good candidates for ultrasound activated reductants.

Chemical compounds which undergo chemical transformation upon application of ultrasound to form quinone compounds can also be used. Such compounds can be considered quinone pro-drugs. These include azobenzene and related azo-dyes, dinitrobenzene and compounds containing a dinitrobezene structure, nitrophenol and compounds containing a nitrophenol structure, phenol, compounds containing a phenolic structure, flavanols, catechol and structures containing a catechol moiety.

The reductant, if administered alone, can be an activator since biological sources of metals, such as iron, exist in the body. Additionally, several reductants are known to mobilize iron from biological stores, such as ferritin, therefore increasing the amount of metal present at the treatment site. Iron is also released during ultrasound exposure by cell lysis during mechanical shearing from ultrasonic cavitation and by degradation of heme compounds during cavitation. The reductant can therefore substantially increase the formation of cytotoxic species. This increase can be further improved by the addition of metals, free bound or chelated, to the body.

For in vivo use, low molecular weight chelators are favored, since they allow easier diffusion of iron into cell walls, where the hydroxyl radical will be generated in close proximity to the polyunsaturated fatty acids and lipids of the cell wall. The hydroxyl radical can therefore initiate and engage in the chain reactions which ultimately lead to hydroperoxide formation. These chelators include classes of compounds recently isolated from wood decay fungi, and have been termed “redox cycling chelators” because of their role in the Fenton mediated degradation of wood by certain fungi. One can readily determine without undue experimentation, if a “wood rot” compound is applicable for use in the present invention by using them in a Fenton reaction. These chelators include phenolate derivatives, glycopeptides, and hydroxamic acid derivatives.

Catechols and other phenolic compounds are also low molecular weight chelants that can be used in the present invention. Several of these compounds also lower the redox potential of the metals with which they interact. It is believed that wood rot fungu use hydroquinone-driven Fenton reactions. For example, 4,5-dimethoxy-1,2-benzenediol and 2,5-dimethoxy-1,4-benezenediol have been isolated from one such fungus and these compounds are believed to chelate iron is a manner that facilitates free radical production by the Fenton reaction. Is was recently discovered that Fenton chemistry is involved in wood rot mechanisms, so other low molecular weight compounds that enhance the Fenton reaction are likely to be isolated from wood rot in the future. These compounds are of interest because their activity will be enhanced when exposed to ultrasound due to the availability of iron during ultrasound exposure as well as the ability of ultrasound to accelerate the Fenton reaction. Quinolines can also be used to enhance the Fenton reaction via chelation.

Oxalate can be used in conjunction with Fenton therapies to increase the rate of production of hydroxyl radicals by preventing ferric iron from reacting with oxygen to form hydro(oxide) complexes.

The chelant can be chosen to modify the hydrophilicity of the metal compound such that it has a longer residence time in the blood. These chelators are commonly used in MRI contrast agents, as disclosed in EP 187947 and WO 89/06979, the entire contents of which patents are hereby incorporated by reference. Using these patents as guides, one skilled in the art can create similar chelated metal compounds which react via Fenton-like mechanisms. Binding the chelant to a macromolecule such as a polysaccharide (e.g., dextran or derivatives thereof) to produce a soluble macromolecular chelant having a molecular weight above the kidney threshold, about 40 kD, ensures relatively long term retention of the contrast agent within the systemic vasculature. Other examples can be found in U.S. Pat. Nos. 4,687,658; 4,687,659, and EP 299975 and EP 130934, the entire contents of which are hereby incorporated by reference.

Vanadium metallocene complexes, such as described in U.S. Pat. No. 6,051,603, can also produce reactive oxygen species via Fenton-type reactions. This patent is hereby incorporated in its entirety by reference.

Fullerene derivatives can be used as metal delivery vehicle when a chelating moiety is attached to the carbon surface. These modified Fullerene compounds can carry up to 30 or more metal atoms. The metal atoms can also be incorporated inside of Fullerenes and Fullerene compounds.

Other metal chelators which can be used in the present invention include but are not limited to citrates, gluconates, succinctness, sulfates, phosphates, tartrates, aluminates, saccharide, lactates, oxalates, formats, fumigates, glycerophosphates, chlorides, ammonium compounds, nitrates, pentonates, sugars, ADP, ATP, PDTA, thiosulfates and thiosulfates, and polymer chelants such as polyvinylpyrollidone and other polyamines. Of these compounds, aminocarboxylates, hydroxcarboxylates, and the biological chelants ADP, ATP, and GTP are preferred because their involvement in radical production is greatly accelerated by ultrasound.

Chelants that increase free radical production when exposed to ultrasound and a metal include aminocarboxylates and their salts, derivatives, isomers, polymers, and iron coordination compounds. Addition of a reducing agent such as ascorbic acid, 1,4-naphthoquinone derivatives, 1,4-benzoquinone derivatives and 1,4-anthraquinone derivatives and/or thiols further increases free radical production. This was demonstrated using the following aminocarboxylate chelants:

Ethylenediaminetetraacetic acid

Ethylene glycol-bis-(2-aminoethyl)-N,N,N′,N′-tetraacetic acid

Diaminocyclohexane-N,N,N′,N′-tetraacetic acid

Nitriloacetic acid

N-2-(hydroxyethyl)ethylenediamine-N,N′,N′-triacetic acid

Diethylenetriaminepentaacetic acid

Picolinic acid

Examples of other aminocarboxylate chelants are diethylenediamine pentaacetic acid, ethylenediaminedisuccinic acid (EDDS), iminodisuccinate (IDSA), methylglycinediacetic acid (MGDA), glutamate, N,N-bis-(carboxymethyl) (GLUDA), diethylenetetraaminepentaacetic acid (DTMPA), ethylenediaminediacetic acid (EDDA), 1,2-bis-(3,5-dioxopiperazine-1-yl)propane (ICRF-187), and N,N′-dicarboxamidomethyl-N,N′-dicarboxylmethyl-1,2-diaminopropane (ICR198). This is not an exclusive list of aminocarboxylate chelants, but is merely presented to illustrate some of the aminocarboxylate chelants that can be used in the present invention.

Chelants that have available a coordination site that is free or occupied by an easily displaceable ligand such as water are preferred. However, this is not a strict requirement for activity.

While any ratio of chelant to metal can be used, generally a ratio of about 0.5:1 to about 10:1 of chelant to metal is preferred.

A number of chelants have been found to increase free radical production when exposed to ultrasound and a metal: hydroxycarboxylate chelants and related compounds, including organic alpha and beta hydroxycarboxylic acid, alpha and beta ketocarboxylic acids and salts thereof, their derivatives, isomers, metal coordination compounds, and polymers.

While a preferred ratio of chelant to metal is about 0.5:1 to about 100:1, a preferred ratio is about 0.5:1 to about 30:1. Addition of a reducing agent such as ascorbic acid, 1,4-naphthoquinone derivatives, 1,4-benzoquinone derivatives, or 1,4-anthraquinone derivatives, and/or thiols used increase radical productions.

Examples of other compounds are tartaric acid, glucoheptonic acid, glycolic acid, 2-hydroxyacetic acid; 2-hydroxypropanoic acid; 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxypropanoic acid; 2,3-dihydroxypropanoic acid; 2,3,4-trihydroxybutanoic acid; 2,3,4,5-tetrahydroxypentanoic acid; 2,3,4,5,6-pentahydroxyhexanoic acid; 2-hydroxydodecanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy -3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid; isocitric acid; agaricic acid; quinic acid; glucuronic acid; glucuronolactone; galacturonic acid; galacturonolactone; uronic acids; uronolactones; dihydroascorbic acid; dihydroxytartaric acid; tropic acid; ribonolactone; gluconolactone; galactonolactone; gulonolactone; mannonolactone; ribonic acid; gluconic acid; citramalic acid; pyruvic acid; hydroxypyruvic acid; hydroxypyruvic acid phosphate; methylpyruvate; ethyl pyruvate; propyl pyruvate; isopropyl pyruvate; phenyl pyruvic acid; methyl phenyl pyruvate; ethyl phenyl pyruvate; propyl phenyl pyruvate; formyl formic acid; methyl formyl formate; ethyl formyl formate; propyl formyl formate; benzoyl formic acid; methyl benzoyl formate; ethyl benzoyl formate; propyl benzoyl formate; 4-hydroxybenzoyl formic acid; 4-hydroxyphenyl pyruvic acid; and 2-hydroxyphenyl pyruvic acid. This list is representative of chelants based on the hydroxycarboxylic acid and ketocarboxylic acid structure but is not all inclusive (Toyokuni (1993)).

The following chelants increase free radical production when exposed to ultrasound and a metal: adenosine diphosphate (ADP), adenosine triphosphate (ATP) and guanosine triphosphate (GTP). In general, a 0.5:1 to 10:1 ratio of chelant to metal is preferred. Addition of a reducing agent such as ascorbic acid, 1,4-naphthoquinone derivatives, 1,4 benzoquinone derivatives, and 1,4-anthraquinone derivatives and/or thiols further increases radical production. We demonstrated this using ADP.

The following compounds increase free radical production when exposed to ultrasound and a metal: phosphonoformic acid, phosphonoacetic acid, and pyrophosphate. In general, a 0.5:1 to 30:1 ratio of compound to metal is preferred. These compounds can act as chelants and/or reducing agents. We demonstrated the activity of these compounds when phosphonoformic acid was added to an iron/EDTA system and radical production was increased.

Addition of a chelant such as aminocarboxylates, hydroxycarboxylates, or biologically relevant chelants such as ADP, ATP, or GTP further increases radical production, particularly when added to the compounds listed in paragraph 0107. Addition of a reducing agent such as ascorbic acid, 1,4-naphthoquinone derivatives, 1,4 benzoquinone derivatives, and 1,4-anthraquinone derivatives and/or thiols further increases radical production particularly when added to the compounds listed in paragraph 0107 (Lindqvist (2001)).

The following compounds increase free radical production when exposed to ultrasound and a metal: tetracycline antibiotics and their derivatives, salts, and polymers. These compounds can act as chelants and/or reducing agents. We demonstrated the activity of these compounds when tetracycline was added to iron and radical production was increased. Examples include but are not limited to methacycline, doxycycline, oxytetracycline, demeclocyline, meclocycline, chlortetracycline, bromotetracycline, daunomycin, dihydrodaunomycin, adriamycin, steffimycin, steffimycin B, 10-dihydrosteffimycin, 10-dihydrosteffimycin B, 13213 RP, tetracycline ref. 7680, baumycin A2, baumycin A1, baumycin B1, baumycin B2, antibiotic MA 144S1, rhodomycin antibiotic complex, musettamycin, antibiotic MA 144L1, aclacinomycin B! antibiotic MA 144 Y, aclacinomycin A, antibiotic MA 144G1, antibiotic MA 144M1, antibiotic MA 144N1, rhodirubin B, antibiotic MA 144U1, antibiotic MA 144G2, rhodirubin A, antibiotic MA 144M2, marcellomycin, serirubicin, oxytetracycline, demeclocycline and minocycline.

Addition of a chelant such as aminocarboxylates, hydroxycarboxylates, or biologically relevant chelants such as ADP, ATP, or GTP further increases radical production, particularly when added with a compound described in paragraph 0109. Addition of a reducing agent such as ascorbic acid, 1,4-naphthoquinone derivatives, 1,4 benzoquinone derivatives, and 1,4-anthraquinone derivatives and/or thiols further increases radical production, particularly in combination with a compound from paragraph 0109 (Quinlan (1998)).

The following compounds increase free radical production when exposed to ultrasound and a metal: hydroxy-1,4-naphthoquinones, their derivatives, isomers, metal coordination compounds, salts, and polymers. These compounds can act as chelants and/or reducing agents. We demonstrated their effectiveness using the following compounds:

10 uM 5-hydroxy-1,4-naphthoquinone
(juglone)
15 uM 2-hydroxy-3-(3-methyl-2-butenyl)-
1,4-naphthoquinone (lapachol)
71 uM 5-hydroxy-2-methyl-1,4-
naphthoquinone (plumbagin)
106 uM 5,8 dihydroxy -1,4-naphthoquinone

Addition of a chelant such as aminocarboxylates, hydroxycarboxylates, or biologically relevant chelants such as ADP, ATP, or GTP further increases radical production. Addition of a reducing agent such as ascorbic acid, 1,4 benzoquinone derivatives, and 1,4-anthraquinone derivatives and/or thiols further increases radical production.

Other examples of hydroxylated 1,4-naphthoquinones include the following compounds and their derivatives: 1,4-naphthalenedione, 2,3-dihydroxy; 1,4-naphthalenedione, 2,5,8-trihydroxy; 1,4-naphthalenedione, 2-hydroxy; 1,4-naphthalenedione, 2-hydroxy-3-(3-methylbutyl); 1,4-naphthalenedione, 2-hydroxy-3-methyl; 1,4-naphthalenedione, 5,8-dihydroxy-2-methyl; alkannin; alkannin dimethylacrylate; aristolindiquinone, chleone A, droserone; isodiospyrin; naphthazarin; tricrozarin A, actinorhodine, euclein, and atovaquone. This list is representative of hydroxy-1,4-naphthoquinones and is not all inclusive.

The following compounds increase free radical production when exposed to ultrasound and a metal: hydroxylated 1,4-benzoquinones, their derivatives, isomers, metal coordination compounds, salts, and polymers. These compounds can act as chelants and/or reducing agents. We demonstrated their effectiveness using the following compound:

Tetrahydroxy 1,4-benzoquinone

Addition of a chelant such as aminocarboxylates, hydroxycarboxylates, or biologically relevant chelants such as ADP, ATP, or GTP further increases radical production, particularly when added with a compound as described above. Addition of a reducing agent such as ascorbic acid, 1,4-naphthoquinone derivatives, and 1,4-anthraquinone derivatives and/or thiols further increases radical production especially in combination with a compound as described above.

Embelin, methylembelin, and rapanone are examples of other hydroxylated 1,4-benzoquinones.

The following compounds increase free radical production when exposed to ultrasound and a metal: hydroxylated anthraquinones, their derivatives, isomers, metal coordination compounds, salts, and polymers. These compounds can act as chelants and/or reducing agents.

Addition of a chelant such as aminocarboxylates, hydroxycarboxylates, or biologically relevant chelants such as ADP, ATP, or GTP further increases radical production especially in combination with a compound as described above. Addition of a reducing agent such as ascorbic acid, 1,4-naphthoquinone derivatives, 1,4 benzoquinone derivatives, and/or thiols further increases radical production more particularly, when and in combination with a compound as described above.

Examples of hydroxylated anthraquinones include but are not limited to the following compounds and their derivatives: alizarin, aloe-emodin, anthragallol, aurantio-obtusin, barbaloin, cascaroside A, cassiamin C, 7-chloroemodin, chrysazin, chryso-obtusin, chrysophanic acid 9-anthrone, digiferrugineol, 1,4-dihydroxy-2-methylanthraquinone, frangulin A, frangulin B, lucidin, morindone, norobtusifolin, obtusifolin, physcion, pseudopurpurin, purpurin, danthron, and rubiadin. Prodrugs such as diacerein that are converted to hydroxylated anthraquinones in the body are also relevant (Gutteridge, et al. (1986); Kagedal, et al., (1999); Lee (1999); Lee, et al. (2001); Muller, et al. (1993)).

Flavonoids such as kaempferol, quercetin, and myricetin and sesquiterpenes such as gossypol and feralin are reducing agents and/or chelants that increase free radical production when exposed to ultrasound and a metal. Addition of a chelant such as aminocarboxylates, hydroxycarboxylates, or biologically relevant chelants such as ADP, ATP, or GTP further increases radical production. Other examples of flavonoids include, but are not limited to acacetin, apigenin, biochanin-A, daidzein, equol, flavanone, flavone, formononetin, genistin, glabranin, liquiritigenin, luteolin, miroestrol, naringenin, naringin, phaseollin, phloretin, prunetin, robinin, and sophoricoside. Derivatives, polymers, and glycosylated forms of these compounds are also relevant. B-dihydroxy and B-trihydroxy flavonoids are preferred (Canada (1990); Laughton. (1989)).

The following compounds increase free radical production when exposed to ultrasound and a metal: anti-tumor antibiotic quinoid agents such as benzoquinones, mitimycins, streptonigrins, actinomycins, anthracyclines, and substituted anthraquinones. These compounds can act as chelants and/or reducing agents.

Free radical production by compounds as described above is enhanced by adding chelants such as aminocarboxylates, hydroxycarboxylates, or biologically relevant chelants such as ADP, ATP, or GTP, or reducing agents such as ascorbic acid or thiols (Gutteridge, et al. (1985); Gutteridge, et al. (1984); Morier-Teissier, et al. (1990)).

The following compounds increase free radical production when exposed to ultrasound and a metal: ascorbic acid, its derivatives, salts and polymers act as ultrasound enhanced reducing agents and/or chelants. Addition of a chelant such as aminocarboxylates, hydroxycarboxylates, or biologically relevant chelants such as ADP, ATP, or GTP further increases radical production (Dognin (1975); Schneider (1988)).

Thiol compounds, their derivatives, and polymers increase free radical production when exposed to ultrasound and a metal. We demonstrated their effectiveness using cysteine as an example of a biological thiol and pennicillamine as an example of a thiol drug. Biological thiols and thiol drugs are preferred. Examples of biological thiols include, but are not limited to cysteinylglycine, cysteamine, thioglycollate and glutathione. Other thiol containing drugs include but are not limited to Captopril, Pyritinol (pyridoxine disulfide), Thiopronine, Piroxicam, Thiamazole, 5-Thiopyridoxine, Gold sodium thiomalate, and bucillamine. In addition, drugs classified as penicillins, cephalosporins, and piroxicam may undergo hydrolytic breakdown in vivo to form thiols; therefore, they are thiol prodrugs. Addition of a chelant such as aminocarboxylates, hydroxycarboxylates, or biologically relevant chelants such as ADP, ATP, or GTP further increases radical production. Addition of a reducing agent such as ascorbic acid, 1,4-naphthoquinone derivatives, 1,4 benzoquinone derivatives, and/or 1,4-anthraquinone derivatives.

A comprehensive list of thiol compounds include 1-(mercaptomethyl)-7,7-dimethylbicyclo[2.2.1]heptan-2-one; 1,2,3-benzotriazine-4(3H)-thione; 1,2-benzisothiazole-3(2H)-thione -1,1-dioxide;1,2-dihydro-3H-1,2,4-triazole-3-thione; 1,2-dihydro-3H-1,2,4-triazole-3-thione and derivatives; 1,2-dihydro-4,5-dimethyl-2H-imidazole-2-thione; 1,3-dihydro-1-methyl-2H-imidazole-2-thione; 1,3-dihydro-2H-naphth[2,3-d] imidazole-2-thione; 1,3-dihydro-4,5-diphenyl-2H-imidazole-2-thione; 1,4-benzoxazepine-5(4H)-thione; 1,4-dihydro-5H-tetrazole -5-thione and derivatives; 1,5-dihydro-4H-pyrazolo[3,4-d]pyrimidine-4-thione; 1,5-dihydro-6H-imidazo[4,5-c]pyridazine-6-thione; 1,7-dihydro-6H-purine-6-thione; 1-adamantanethiol; 2(1H)-benzimidazolinethione; 2,4-diamino-6-mercapto-1,3,5-triazine; 2,4-dimethylbenzenethiol; 2,5-dimethylbenzenethiol; 2,6-dimethylbenzenethiol; 2-adamantanethiol; 2-amino-1,7-dihydro-6H-purine-6-thione; 2H-1,4-benzothiazine-3(4H)-thione; 2-imidazolidinethione; 2-Isopropyl-3-methylbenzenethiol; 2-isopropyl-4-methylbenzenethiol; 2-isopropyl-5-methylbenzenethiol; 2-mercapto-4H-1-benzopyran-4-thione; 2-mercapto-5-methyl-1,3,4-thiadiazole; 2-mercapto-5-nitrobenzimidazole; 2-mercaptothiazoline; 2-methyl-1-propenethiol; 2-methylene-1,3-propanedithiol; 2-propene-1-thiol; 3,4-dihydro-4,4,6-trimethyl-1-(4-phenyl-2-thiazolyl)-2(1H)-pyrimidinethione; 3,4-dihydro-4,4,6-trimethyl-2(1H)-pyrimidinethione; 3-amino -5-mercapto-1H-1,2,4-triazole; 3-bromo-1-adamantanethiol; 3-mercapto-5-methyl-1,2,4-triazole and derivatives; 3-mercaptocyclohexanone and derivatives; 3-quinuclidinethiol; 3-thio-9,10-secocholesta-5,7,10(19)-triene; 4-amino-2,4-dihydro -5-phenyl-3H-1,2,4-triazole-3-thione; 4-amino-3-hydrazino-5-mercapto-1,2,4-triazole; 4-benzocyclobutenethiol; 4-biphenylthiol; 4-Isopropyl-2-methylbenzenethiol; 5,6-dichloro -2-mercapto-1H-indole; 5′-amino-2′,3,3′,4-tetrahydro-4,4,6-trimethyl-2,2′-dithioxo[1(2H),4′-bipyrimidin]-6′(1′H)-one; 5-isopropyl-2-methylbenzenethiol; 5-mercapto-3-methyl-1,2,4-thiadiazole; 6-amino-2-mercaptopurine; 6-thioinosine; 7-(nercaptomethyl)-1,7-dimethylbicyclo[2.2.1]heptan-2-one; 7-mercapto-3H-1,2,3-triazolo[4,5-d]pyrimidine; Azothiopyrine; benzo[c]thiophene-1(3H)-thione; bis(1-methylethyl)carbamothioic acid S-(2,3,3-trichloro-2-propenyl) ester; Caesium [2,6-bis(2,4,6-triisopropylphenyl)phenyl]thiolate; (3β)-cholest-5-ene-3-thiol; Cyclohexanethione; Lithium [2,6-bis(2,4,6-triisopropylphenyl)phenyl]thiolate; naphtho[1,2-d]thiazole -2(1H)-thione; naphtho[2,1-d]thiazole-2(3H)-thione; phenylmethanethiol; Potassium [2,6-bis(2,4,6-triisopropylphenyl)phenyl]thiolate; Rubidium [2,6-bis(2,4,6-triisopropylphenyl)phenyl]thiolate; Sodium [2,6-bis(2,4,6-triisopropylphenyl)phenyl]thiolate (Diez (2001)).

Sodium sulfide and sodium sulfite are reducing agents that increase free radical production when exposed to ultrasound and a metal. We demonstrated this using sodium sulfite. Addition of a chelant such as aminocarboxylates, hydroxycarboxylates, or biologically relevant chelants such as ADP, ATP, or GTP further increases radical production.

The terms “a receptor” and “an antigen” refer to a chemical group in a molecule which comprises an active site in said molecule, or to an array of chemical groups in a molecule which comprise one or more active sites in the molecule, or to a molecule comprised of one or more chemical groups or one or more arrays of chemical groups, which group or groups or array of groups comprise one or more active sites in the molecule. An “active site of a receptor” has a specific capacity to bind to or has an affinity for binding to a vector. With respect to use with the term “a receptor” or with the term “active site in a receptor”, the term “vector” as used herein refers to a molecule comprised of a specific chemical group or a specific array of chemical groups receptor recognizing group, which molecule, group, or array of groups is complementary to or has a specific affinity for binding to a receptor, especially to an active site in a receptor, to which otherwise modifies the biodistribution of the overall composition of matter in a desired manner. Examples include cell surface antigens, cell surface and intracellular receptors which bind hormones, and cell surface and intracellular receptors which bind drugs. Sites of specific association of specific hormone binding to cellular receptors and specific binding of drugs or cellular receptors are examples of active sites of the receptors, and the hormones or the drugs are examples of vectors for the respective receptors.

The vector group can be selected from a wide variety of naturally occurring or synthetically prepared materials, including but not limited to enzymes, amino acids, peptides, polypeptides, proteins, lipoproteins, glycoproteins, lipids, phospholipids, hormones, growth factors, steroids, vitamins, polysaccharides, lectins, toxins, nucleic acids (including oligonucleotides), haptens, avidin and derivatives thereof, biotin and derivatives thereof, antibodies (monoclonal and polyclonal), anti-antibodies, antibody fragments and antigenic materials (including proteins and carbohydrates). The vector group can also be components or products of viruses, bacteria, protozoa, fungi, parasites, rickettsia, molds, as well as animal and human blood, tissue and organ compositions. The vector group can also be a pharmaceutical drug or synthetic analog of any of the materials mentioned above, as well as others known to one skilled in the art. Additional specific vector groups are described in WO 96/40285, the entire contents of which are hereby incorporated by reference.

Preferred vectors are antibodies and various immunoreactive fragments thereof, proteins and peptides, as long as they contain at least one reactive site for reacting with a vector reactive group or with linking groups. The site can be inherent to the vector or it can be introduced though appropriate chemical modification of the vector. The antibodies and fragments thereof can be produced by any conventional means, including molecular biology, phage display, and genetic engineering.

The term “antibody fragments” refers to a vector which comprises a residue of an antibody, which characteristically has an affinity for binding to an antigen. Antibody fragments exhibit at least a percentage of affinity for binding to an antigen, this percentage being in the range of 0.001 percent to about 1000 percent, preferably about 0.1 percent to about 1000 percent, of the relative affinity of the antibody for binding to the antigen.

Additional preferred vectors are peptides, oligopeptides, or peptoids, which vectors are composed of one or more amino acids whose sequence and composition comprise a molecule, specific chemical group or a specific array of chemical groups, which are complementary to or have a specific affinity for binding to a receptor, especially to an active site of a receptor. Especially preferred vectors are peptidomimetic molecule, which are fully synthetic organic materials that are the structural or functional equivalent of receptor groups derived or identified form antibodies, antibody fragments, proteins, fusion proteins, peptides, or peptoids, and that have affinity for the same receptor. Other peptidometric vectors include chemical entities such as drugs, for example, which show affinity for the receptor, and especially for the active site of the receptor of interest.

Peptidometric vectors can be identified using molecule biological techniques such as protein mutation, phage display, genetic engineering, and other such techniques know to those skilled in the art.

The ultrasound transducer used in sonodynamic therapy may be applied externally or may be implanted. It can be introduced into the body via endoscopy or catheter.

Focused ultrasound can be guided by imaging modalities, such as MRI. The applied ultrasound can act as both the irradiation source and as an imaging modality.

The exact operating parameters for photodynamic therapy and sonodynamic therapy are determined depending upon the specific irradiation system being used, as well as on the target tissue or other application.

For purposes of the present invention, “a metal” means an element that forms positive ions when its compounds are in solution and whose oxides form hydroxides rather than acids with water. Metals occur in every group of the periodic table except VIIA and the noble gas group.

The preferred metals for use in the present invention are transition metals, lanthamides, and actinides. The metal, can be in the form of free metal ions, metal salts (inorganic or organic), metal oxides, metal hydroxide, metal sulfides, coordinate compounds, or clathrates. The metal can be present in one or more oxidations states. A combination of different metals can be used in combination or sequentially. These metals may be bound, covalently or noncovalently, to complexing or chelating agents, including lipophilic derivatives thereof, or to proteinaceous macromolecules. The metals can be incorporated into liposomes or vesicles. Polymerized and particulate forms of metals can also be used. Biological sources of iron, such as ferritin and transferrin, can also be used. Metals and metal compounds that are used as MRI contrast agents can also be used. Typical MRI contrast agent compositions are described in U.S. Pat. Nos. 6,088,613; 5,861,140; 5,820,851; 5,534,241; 5,460,700; 5,411,730; 5,409,689; 5,407,657; 5,336,762; 5,314,679; 5,242,681; 5,236,915; 5,336,695; 5,213,788; 5,155,215; 5,120,527; 5,055,288; and SO 30688A2, the entire contents of with which are hereby incorporated by reference.

Sonotherapeutic delivery systems, with which generally involve rupturing drug filed microspheres at the desired site by application of ultrasound energy, are suitable delivery vehicles for the sonotherapeutic agents and/or metals. These delivery systems are described in detail in U.S. Pat. Nos. 6,028,066; 5,997,898; 6,039,967; PCT applications 991391A1, 9851284A1, 9842384A1, 0012062A1, 9939697A1; European applications 988061A1, 981333A1, 959908A1, 831932A1, 0097907A1; and Japanese application 10130169A.

Sonodynamic or photodynamic delivery systems can be in the form of a microsphere containing the sonodynamic agent in which the activator metal is covalently or non-covalently attached to the surface or components of the microsphere. Two types of microspheres, one containing the sonodynamic agent and one containing the activator, can be used in combination or sequentially.

The sonodynamic or photodynamic agent and metal activator can be combined via covalent or non-covalent bonds. In a preferred embodiment, this is achieved by attaching the sonodynamic agent to the surface of ferritin or modified ferritin through ionic or covalent attachments.

In one embodiment of the invention, the activator may include a molecule with which is detectable via an in vivo diagnostic imaging modality, such as X-ray, MRI, ESR, NMR, ultrasound, light imaging scintigraphy, in vivo microscopy such as confocal microscopy, photoacoustic imaging and acousto-optical imaging, visual observation, photographic imaging, magnetotomography, or electrical impedance tomography. The metal activator itself is suitable for MRI imaging, permitting simultaneous treatment and imaging.

The metal activator can include a moiety to modify its biodistribution, thus targeting the desired location with greater specificity. Examples of these moieties include antibodies, antibody fragments, proteins, and oligopeptides which have an affinity for cell surface receptors, particularly receptors associated with surfaces of diseased or rapidly proliferating cells, and peptides and non-peptide drugs with which are preferentially taken up by diseased or rapidly proliferating cells. These targeting moieties also include tumor-targeting drug compound, blood residence prolonging compounds, folic acid and derivatives thereof. Activators with which contain sulfonic acid groups of derivatives thereof promote retention at tumor sites.

The metal activator can be administered prior to administering the sonodynamic or photodynamic agent, or in combination with the sonodynamic or photodynamic agent. Different routes may be used for administering the metal activator and the sonodynamic or photodynamic agent. Dosage

For photodynamic therapy or sonodynamic therapy, the photodynamic and/or sonodynamic compound is administered in conjunction with at least one activator. The dosage used will depend on the mode of administration, the nature of the condition being treated, the patient's size and species. Where a reporter is used, the dosage also depends on the nature of the imaging modality and the nature of the reporter. Where the reporter is a non-radioactive metal ion, generally dosages of about 0.001 to about 5.0 moles of chelated imaging metal ion per kilogram of patient body weight are effective to achieve adequate contrast enhancements.

The photodynamic or sonodynamic compounds plus activator according to the present invention may be administered by any convenient route, such as by injection or infusion into muscle, tumor tissue, or the vasculature, subcutaneously, or interstitially, by administration into an eternally voiding body cavity such as into the digestive tract (orally or rectally), vagina, uterus, bladder, ears, nose or lung, by transdermal administration by iontophoresis or by topical application, or by topical application to a surgically exposed site. Direct injection into a tumor is one preferred administration route.

The administration forms used may be any conventional form for administration of pharmaceuticals, such as solutions, suspensions, dispersions, syrups, powders, tablets, capsules, sprays, creams, gels, and the like. Oral administration of photodynamic or sonodynamic compounds plus metal activators is often preferred because of enhanced patient compliance and ease of administration. While not every agent is bioavailable by this route, since not all molecules are chemically stable in the environs of the gut, transportable across alimentary membranes for absorption into the blood/lymphatics, or active even if accessible due to metabolic processes within the gut or possible solubility issue. However, it is also known that alteration of the molecular structure to control the relative hydrophobicity of the molecule within a preferred range can increase the oral availability of the agent.

Any known route of administration of drugs or agents to mammals are envisaged by the present invention.

The photodynamic or sonodynamic compounds can be formulated with conventional pharmaceutical or veterinary aids, such as emulsifiers, fatty acid esters, gelling agents, stabilizers, antioxidants, osmolality adjusting agents, buffers, pH adjusting agents, etc., and may be in a form suitable for parenteral or enteral administration. Thus, the photodynamic or sonodynamic compounds of the present invention, which may be formulated with the metal activator or administered separately from the metal activator, can be in conventional pharmaceutical administration forms such as tablets, capsules, powders, solutions, suspensions, dispersions, syrups, suppositories, etc.

To treat patients according to the present invention, the sonodynamc therapy may be effected by exposing the patient to an effective amount of ultrasound acoustic energy as described in the literature. Generally, frequency and power levels that produce ultrasonic cavitation or mechanical shearing in the body are preferred. Generally, this will involve exposure to focused ultrasound, e.g., at a power level of about 0.1 to about 20 Wcm⁻², preferably about 4 to about 12 Wcm⁻², a frequency of about 0.01 to about 10.0 MHZ, preferably about 0.1 to about 5.0 MHZ, particularly about 0.001 to about 2.2 MHz, for periods of 10 milliseconds to 60 minutes, preferably for about one second to about five minutes. As one skilled in the art can readily appreciate, these values depend on the transducer frequency, type of tissue irradiated, and sonodynamic agent used, and these values are merely illustrative. The important characteristic is that mechanical shearing and/or cavitation are required for treatment

Particularly preferably, the patient is exposed to ultrasound at an acoustic power of about 5 mW to 10 W with a fundamental frequency of about 0.01 to about 1.2 MHZ and a corresponding second harmonic frequency, as this produces the exposure necessary to achieve a cytopathogenic effect.

“Treatment” or “treating” means any treatment of a disease in a mammal, including:

preventing the disease, i.e., preventing the clinical symptoms of the disease from developing;

inhibiting the disease, i.e., arresting the development of clinical symptoms; and/or

relieving the disease, i.e., causing the regression or disappearance of clinical symptoms.

Photodynamic and Sonodynamic Therapy

For photodynamic therapy, the parameters of the pulse of light required for activation of the photosensitizable compound may be determined empirically, for example, by direct measurement of the fluorescence activity of the sensitizer plus activator under different irradiation regimes, or by measuring the slope of effect evoked on final subtract of the sensitizer activity under different radiation regimes which change can be easily determined by a fluorescence or activity effect on a substrate.

It should be noted that there exists an inverse relationship between the intensity of irradiation and the duration, i.e., the lower the intensity above the threshold of activation, the longer the duration should be. Therefore, for each specific photosensitizable compound, there exist several pulses which can be used for treatment purposes.

For sonodynamic therapy, ultrasound or any other externally controllable sonic energy source is administered, the toxicity of which is selectively enhanced by a sensitizer.

The preferred sonodynamic agent employed in the present invention is ultrasound, particularly low intensity, non-thermal ultrasound, i.e., ultrasound generated within the wavelengths of about 0.1 MH and about 5.0 MHZ and at intensities between about 3.0 and about 5.0 W/cm². Ultrasound can be generated by a focused array transducer, driven by a power amplifier. The diameter of the focused array transducer varies in size and spherical curvature to allow for variation of the focus of the ultrasonic output. Commercially available therapeutic ultrasound devices can be used. Frequency and power levels that produce ultrasonic cavitation or mechanical shearing in the body are preferred.

The photodynamic or sonodynamic compounds may be used alone or in any desired combination of photodynamic or sonodynamic compounds. Where there is a plurality of photodynamic or sonodynamic compounds, they may be administered separately, sequentially, or simultaneously. The metal activator can be administered separately, sequentially, or simultaneously with the photodynamic or sonodynamic compounds.

Sonodynamic or photodynamic therapy using a sonodynamic or photodynamic agent along with a metal enhancer can be used for all types of therapy for which sonodynamic and/or photodynamic therapy can be used. For example, patients can be treated according to the present invention to induce apoptosis or programmed cell death thereby to prevent and/or treat a variety of diseases or conditions and provide a variety of benefits. Cancer can be prevented by applying ultrasound energy or light energy along with an enhancer and a metal to induce apoptosis or programmed cell death of precancerous cells in different tissues and organs of a mammal.

Additionally, cancer cells can be exposed to ultrasonic or light energy along with an enhancer and a metal in an amount effective to induce apoptosis of cancer cells. The present invention can be used to induce apoptosis undergoing abnormal proliferation in target cells having one or more growth factors including, but not limited to, EGF, TGF, NGF, FGF, IFG, and PDGF.

The present invention can also be used to affect cells undergoing other types of abnormal proliferation, such as, for example, in conditions including arteriosclerosis, vascular and fibrotic proliferative diseases, retinopathies, eczema or psoriasis, by applying sound and/or light energy along with an enhancer and a metal.

Apoptosis is a general property of most cells, being fundamental for the organization and life span of any organism to control homeostasis and cell populations. It is necessary to achieve an adequate balance between the sufficient survival of cells and overwhelming proliferation and expansion. This is of particular importance in preventing and treating malignant growth, but is also necessary to limit expansion of immune cells challenged by pathogens or other stimuli, and as a defense mechanism to remove self-reactive lymphocytes. In aging cells and/or tissues that exhibit functional deficiencies, apoptosis is a useful approach for increasing the turnover of senescent cells and thus trigger the renewal of cellular function and structure.

Accordingly, sonodynamic or photodynamic therapy according to the present invention is effective in treating conditions characterized by neoplastic tissue, including the cancers sarcoma, lymphoma, leukemia, carcinoma and melanoma; cardiovascular diseases such as arteriosclerosis, atherosclerosis, intimal hyperplasia and restenosis; and other activated macrophage-related disorders including autoimmune diseases such as rheumatoid arthritis, Sjogrens scleroderma, systemic lupus erthematosis, non-specific vasculitis, Kawasaki's disease, psoriasis, Type I diabetes, and pemphigus vulgaris. Other diseases and conditions that can be treated by the process of the present invention include granulomatous diseases such as tuberculosis, sarcoidosis, lymphomatoid granulomatosis, and Wegner's granulomatosis; inflammatory diseases such as inflammatory lung diseases such as interstitial penumonitis and asthma; inflammatory bowel disease such as Crohn's disease; inflammatory arthritis, and in transplant rejection, such as in heart/lung transplants. Additional treatment options include cervical dysplasia and cervical ablation, endometriosis and endometrial ablation, fibroids, treatment of diseased tissues after surgery (e.g., treating tissue surrounding a tumor after its surgical removal), bone marrow purging to remove tumor cells that may contaminate bond marrow during autologous bone marrow transplants, prostate cancer and benign prostate hyperplasia (BPH), age-related macular degeneration (AMD), and for immunomodulation (e.g., to suppress development of contact hypersensitivity, abrogate development of acute adjuvant enhanced arthritis, and prolong survival of skin allografts). Cosmetic treatments are also included, such as removal of skin discoloration, moles, birthmarks, spider and varicose veins, and unwanted hair. The parameters of the pulse (light, ultrasound, microwave, etc.) required for activation of the photosensitizable or sonosensitizable compound in the presence of at least one metal can be determined empirically, for example by direct measurement of the fluorescence activity of the sensitizer under different irradiation regimes, or by measuring the slope of effect on the effect of sensitizer activity under different radiation regimes, which change may be easily determined by a fluorescence or activity effect on a substrate. The parameters of energy irradiation which are sufficient to terminate or significantly reduce the change in fluorescence can be used in accordance with the present invention.

It is also possible to combine photodynamic therapy with sonodynamic therapy for enhanced effect of each therapy. In this case, a patient is treated with a sonodynamic compound and exposed to sound waves, as well as with a photodynamic compound and exposed to light waves. Because the activator enhances both the sonodynamic compound and the photodynamic compound, only one activator need be administered for both forms of treatment. However, if one activator is more effective than another activator in photodynamic therapy as opposed to sonodynamic therapy, then a combination of activators may be administered.

Ultrasound according to the present invention can also be used to induce hemostasis, particularly following an automobile accident which internal organs are damaged and endoscopic fibers or catheters cannot be used to treat ruptured organs or intra-liver bleeding. Moreover, bleeding gastric ulcers or ruptured esophageal varices can be treated by the method of the present invention. In this embodiment, a sonodynamic agent is introduced to the body along with a metal activator. Ultrasound energy is applied at a selected site in the body at a frequency sufficient to create hemostasis. This embodiment is particularly useful immediately after bleeding has begun, so that bleeding can be halted while the patient is being transported in an ambulance to an urgent care center. Death rates from trauma are lowered by temporarily stopping the bleeding until major surgical intervention can be performed in a hospital.

Deep locations of the body including but not limited to the liver, abdominal aorta, and their bleeding organs can be treated to halt bleeding without surgical intervention. Because ultrasound energy is used, body organs and structure are not damaged.

In the present invention, photodynamic and sonodynamic agents are combined with an activator, followed by irradiation of the activator-agent combination. In one embodiment of this invention where the activator is a transition metal, the photodynamic and sonodynamic agents are preferably capable of chelation with a metal, i.e., the metal ion is attached by coordinate links to two or more non-metal atoms in the same molecule. Generation of Free Radicals for Chemical Reactions

Free radicals are reactive chemical species possessing a free (unbonded or unpaired) electron. Radicals may also be positively or negatively charged species carrying a free electron (ion radicals). Free radicals are very reactive chemical intermediates and generally have a short lifetime, generally a half-life of less than 10-3 seconds.

Once they are formed, radicals undergo two types of reactions: propagation reactions and termination reactions. In propagation, a radical reacts to form a covalent bond and to generate a new radical. Three of the most common propagating reactions are atom abstractions, beta-scission, and addition to carbon-carbon double bonds or aromatic rings. In a termination reaction, two radicals interact in a mutually destructive reaction in which both radicals form covalent bonds and the reaction terminates. The two most common termination reactions are coupling and disproportionation.

Radical chain reactions are involved in many commercial processes, including polymerization and copolymerization, polymer crosslinking, and polymer degradation. Other radical-initiated polymer processes include curing of resins or rubber, grafting of vinyl monomers onto polymer backbones, and telomerizations.

Radical reaction initiation with ultraviolet radiation is widely used in industrial processes. This process generally requires the presence of a photoinitiator. According to the present invention, however, visible light as well as ultraviolet or other types of light can be used in connection with a photoinitiator and a metal to generate free radicals.

Free-radical polymerization can be conducted in a variety of ways, including bulk polymerization, solution polymerization, suspension polymerization, and emulsion polymerization.

For generation of free radicals, a photoinitiator and/or sononinitiator plus a metal is subjected to the appropriate wavelength of light or sound for an appropriate amount of time. The free radicals thus generated are used for initiating and accelerating a variety of reactions, as described above.

Any conventional sonodynamic or photodynamic agents can be used in the present invention along with a metal to enhance their sonodynamic or photodynamic effect.

Conventional sonodynamic agents include the following classes of compounds:

1. Porphyrins, comprising four pyrrole rings together with four nitrogen atoms and two replaceable hydrogen atoms, for which various metal atoms can be readily substituted. Porphyrins include hemins, chlorophylls, and cytochromes. Specific porphyrins used include gallium porphyrin, porphyrin analogs and derivatives, mesoporphyrin, proptoprohyrin, and hematoporphyrin;

2. Texaphyrins, aromatic pentadentate macrocyclic expanded porphyrins, also described as an aromatic benzannulene containing both 18 pi and 22 pi electron delocalization pathways;

3. Cyanines and phthalocyanines, dyes consisting of two heterocyclic groups connected by a chain of conjugated double bonds containing an odd number of carbon atoms. Cyanines include isocyanines, merocyanines, cryptocyanines, and dicyanines. Phthalocyanines are any group of benzoporphyrins which comprise four isoindole groups joined by four nitrogen atoms;

4. Chromophores, compounds which absorb and/or emit light, particularly those with delocalized electron systems. Chromophores can alternatively contain a complexed metal ion. The term includes fluorophores as well as phosphorescent compounds. A more complete listing of chromophores can be found in WO9852609, the entire contents of which are hereby incorporated by reference;

5. Water soluble polymers (hexamers and higher polymers), particularly polyalkyleneoxide compounds such as those described in WO9852609, the entire contents of which are hereby incorporated by reference. The sensitizer agent is selected form the group consisting of water soluble polymers and derivatives thereof, surfactants, oil-in-water emulsions, stabilized particles, and chromophoric groups such as sulfonated dyes. Preferably the sensitizer agent is a water soluble polymer such as a polyalkylene oxide or a derivative thereof;

6. DMSO (dimethylsulfoxide) and DMF (dimethylformamide);

7. Chemotherapeutic compounds such as adriamycin and derivatives thereof, mitomycin and derivatives thereof, diazaquinone, and amphotercin;

8. Chlorines, pheophorbide, acridine orange and acridine derivatives, methylene blue, fluorescein, neutral red, rhodamins, Rose-Bengal, tetracycline, and purpurins;

9. Antioxidants, such as vitamin E, N-acetylcysteine, glutathione, vitamin C, cysteine, methionine, 2-mercaptoethanol, and/or photosensitizing molecules. A complete listing is provided in U.S. Pat. No. 5,984,882, the entire contents of which are hereby incorporated by reference.

10. Xanthene dyes.

11. Hypericine, hypocrellins, and perylenequinones. Examples can be found in WO 02/34708 and WO 98/33470, the entire contents of which are hereby incorporated by reference.

The hypocrellin derivates of WO 02/34708 consist of amino-substitued demethoxylated hypocrellins A and B, whose structures are shown as V and VI:

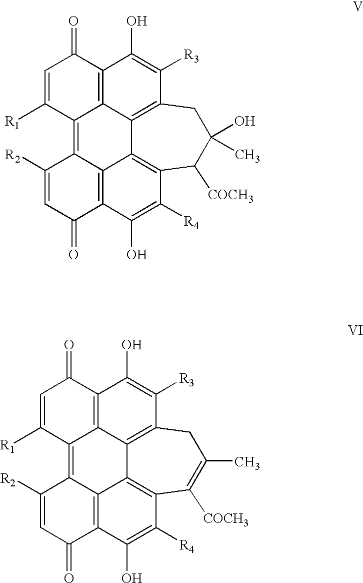

where R₁, R₂, R₃, R₄ are OCH₃ or NHCH₂Ar (Ar are phenyl or pyridyl group), NHCH(CH₂)ₙ where —CH(CH₂)ₙ are alicyclic group and N=3, 4, 5, 6). 2-BA-2-DMHB is where R₁, R₂, R₃ are OCH₃, and R₄ is NH(CH₂)₃CH₃. Alternatively, R₁, R₂, R₃, R₄ may be OCH₃ or NHCH₂(CH₂)ₙAr, wherein Ar is a phenyl, naphthyl, polycyclic aromatic or a heterocyclic moiety, and n is 0-12.

These hypocrellin derivatives also include 2-butylamino-2-demethoxy-hypocrellin B (2-BA-2-DMNB), which exhibits strong absorption in the red spectral region. Compared with its parent compound HB its absorption bands extended toward longer wavelengths. Substituted perylenequinones as described in WO 98/33470 include:

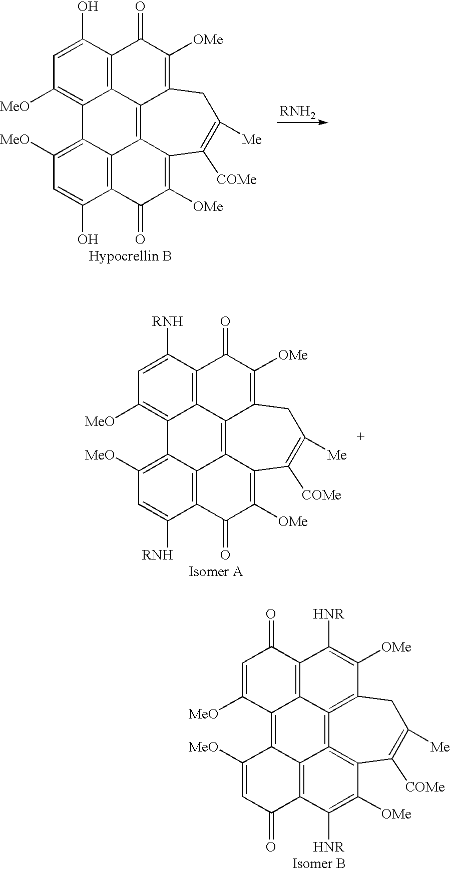

Conventional photodynamic agents include texaphyrins, porphyrins, phthalocyanines, chlorine, rhodamine derivatives as described above for sonodynamic agents.

Additional photodynamic agents include precursors to porphyrin such as 5-aminolevulinic acid; benzophenoxazine analogs; chlorophyll and conjugates of chlorophyll and bacteriochlorophyll derivatives with amino acids, peptides and proteins; porphycenes; pyrylium compounds; thiopyrylium compounds; selenopyrylium compounds; telluropyrylium compounds; fullerene derivatives; phylloerythrins; pyropheophorbides; boron difluoride compounds; ethylene glycol esters substituted perylenequinones; 1, 3, 4, 6-tetrahydroxyhelianthrone and its derivatives; quinolines; thiazine dyes; polycyclic quinines; and other biocompatible chromophores capable of cytotoxic effects upon irradiation with light waves.

The following nonlimiting examples will further describe the present invention.

### EXAMPLE 1

The following example uses the thiobarbituric acid-reactive substances (TBARS) assay to identify chelants that enhance radical production during ultrasound exposure. All solutions were prepared in pH 7.5 phosphate buffer containing approximately 2 mM deoxyribose, 0.01% hydrogen peroxide, 0.025 mM ferrous iron, and 0.03-0.04 mM chelant. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure is determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Chelant | Control |
| No chelant | 19% |
| Desferrioxamine mesylate | 92% |
| Nitriloacetic acid | 69% |
| Ethylenediaminetetraacetic acid | 64% |
| Diaminocyclohexane-N,N,N′,N′- | 61% |
| tetraacetic acid | |
| N-(2- | 34% |
| Hydroxyethyl)ethylenediamine- | |
| N,N′,N′-triacetic acid | |
| Ethylene glycol-bis(2- | 29% |
| aminoethyl)-N,N,N′,N′- | |
| tetraacetic acid | |

### EXAMPLE 2

The following example uses the thiobarbituric acid-reactive substances (TBARS) assay to identify chelants that enhance radical production during ultrasound exposure. All solutions were prepared in pH 7.5 phosphate buffer containing approximately 2 mM deoxyribose, 0.01% hydrogen peroxide, 0.02-0.03 mM ferric iron, and 0.04-0.05 mM chelant. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure was determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Chelant | Control |
| No chelant | 0% |
| Ethylenediaminetetraacetic acid | 575% |
| Ethylene glycol-bis(2- | 520% |
| aminoethyl)-N,N,N′,N′- | |
| tetraacetic acid | |
| Diaminocyclohexane-N,N,N′,N′- | 446% |
| tetraacetic acid | |
| Nitriloacetic acid | 238% |
| N-(2- | 224% |
| Hydroxyethyl)ethylenediamine- | |
| N,N′,N′-triacetic acid | |
| Diethylenetriaminepentaacetic | 177% |
| acid | |
| Desferrioxamine mesylate | 81% |

### EXAMPLE 3

The following example uses the thiobarbituric acid-reactive substances (TBARS) assay to identify chelants that enhance radical production during ultrasound exposure. All solutions were prepared in pH 7.5 phosphate buffer containing approximately 2 mM deoxyribose, 0.01% hydrogen peroxide, 0.025 mM ferrous iron, and 0.07-0.11 mM chelant. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure was determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Chelant | Control |
| No chelant | 19% |
| Picolinic Acid | 53% |
| 3-(2-Pyridyl)-5,6-bis(5-sulfo- | 50% |
| 2-furyl)-1,2,4-triazine | |
| (ferene) | |
| 3-(2-Pyridyl)-5,6-diphenyl- | 45% |
| 1,2,4-triazine-4¢,4¢¢- | |
| disulfonic acid (ferrozine) | |
| 1,10 Phenanthroline | 33% |
| Citrate | 31% |
| Adenosine diphosphate (ADP) | 26% |

### EXAMPLE 4

The following example uses the thiobarbituric acid-reactive substances (TBARS) assay to identify chelants that enhance radical production during ultrasound exposure. All solutions were prepared in pH 7.5 phosphate buffer containing approximately 2 mM deoxyribose, 0.01% hydrogen peroxide, 0.02-0.03 mM ferric iron, and 0.07-0.11 mM chelant. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure was determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Chelant | Control |
| No chelant | 0% |
| Adenosine diphosphate (ADP) | 280% |
| 3-(2-Pyridyl)-5,6-bis(5-sulfo- | 207% |
| 2-furyl)-1,2,4-triazine | |
| (ferene) | |
| Picolinic Acid | 175% |
| Citrate | 161% |
| 3-(2-Pyridyl)-5,6-diphenyl- | 136% |
| 1,2,4-triazine-4,4-disulfonic | |
| acid (ferrozine) | |

### EXAMPLE 5

The following example uses the thiobarbituric acid-reactive substances (TBARS) assay to identify compounds that enhance radical production during ultrasound exposure of solutions containing iron or iron plus a chelant. All solutions were prepared in pH 7.5 phosphate buffer containing approximately 2 mM deoxyribose, 0.01% hydrogen peroxide, 0.08-0.1 mM ferric iron, and the additives indicated in the table below. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure was determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Additive | Control |
| No additive | 76% |
| EDTA (0.15 mM) | 277% |
| Foscarnet (phosphonoformic | 380% |
| acid) (0.15 mM) + EDTA (0.15 | |
| mM) | |

### EXAMPLE 6

The following example uses the thiobarbituric acid-reactive substances (TBARS) assay to identify metals that enhance radical production during ultrasound. All solutions were prepared in pH 7.5 phosphate buffer containing approximately 2 mM deoxyribose, 0.01% hydrogen peroxide, and the additives indicated in the table below. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure was determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Additive | Control |
| No additive | 0% |
| Ferrous iron added as Fe(NH₄)₂(SO₄)₂ (0.05 | 34% |
| mM) + ferric iron added as FeCl₃ (approx | |
| 0.05 mM) | |
| Ferrous iron added as Fe(NH₄)₂(SO₄)₂ (0.1 mM) | 12% |
| Ferric iron added as FeCl₃ (approx 0.1 mM) | 76% |
| Ferritin (approx. 0.2 mg/mL) | 6% |
| Ferrous iron added as Fe(NH₄)₂(SO₄)₂ (0.05 | 253% |
| mM) + ferric iron added as FeCl₃ (approx | |
| 0.05 mM) + 0.15 mM EDTA | |
| Ferrous iron added as Fe(NH₄)₂(SO₄)₂ (0.1 mM) + | 106% |
| 0.15 mM EDTA | |
| Ferric iron added as FeCl₃ (approx 0.1 mM) + | 277% |
| 0.15 mM EDTA | |
| Ferritin (approx. 0.2 mg/mL) + EDTA (0.15 | 18% |
| mM) | |
| Cupric chloride (0.026 mM) | 82% |

### EXAMPLE 7

The following example uses the thiobarbituric acid-reactive substances (TBARS) assay to show the effect of chelant concentration on the enhancement of radical production during ultrasound exposure. All solutions are prepared in pH 7.5 phosphate buffer containing approximately 2 mM deoxyribose, 0.01% hydrogen peroxide, 0.025 mM ferrous iron, approximately 0.025 mM ferric iron, and the ratio of chelant to combined iron indicated in the table below. Solutions are sonicated at 30 W, 2 MHz, 32-34 degrees Celsius for ten minutes using a PZT-8 1.8 cm diameter custom transducer. The sonicated solution is placed on an orbit shaker rotating at 25 RPM to ensure even sonication of the solution while the transducer is held stationary. Control solutions are placed in a controlled temperature bath at 32-34 degrees Celsius without sonication. After 10 minutes of treatment, 1 mL test solution is placed in a test tube followed by 2 mL of 1% 2-thiobarbituric acid and 2 mL of 2.8% trichloroacetic acid. The test tube is sealed and heated to 90 degrees Celsius for 30 minutes and allowed to cool to room temperature for 20 minutes. The absorbance at 532 nm is measured. The enhanced radical production during ultrasound exposure is determined by comparing the amount of deoxyribose degradation that occurs in the sonicated solution versus the control solution using the following equation:

$$\text{\% activity} = \frac{{Abs}_{532} sonicated solution - {Abs}_{532} control solution}{{Abs}_{532} control solution} \times 100$$

Results:

| | Approximate |
|---|---|
| | Chelant:Iron Ratio |
| | for Optimum |
| | Ultrasound Mediated |
| Chelant | Activity vs Control |
| Desferrioxamine mesylate | 1:1 to 1:10 |
| Nitriloacetic acid | 1:1 to 1:10 |
| Ethylenediaminetetraacetic | 1:1 to 1:10 |
| acid | |
| Diaminocyclohexane-N,N,N′,N′ - | 1:1 to 1:10 |
| tetraacetic acid | |
| N-(2- | 1:1 to 1:10 |
| Hydroxyethyl)ethylenediamine- | |
| N,N′,N′-triacetic acid | |
| Ethylene glycol-bis(2- | 1:1 to 1:10 |
| aminoethyl)-N,N,N′,N′- | |
| tetraacetic acid | |
| Diethylenetriaminepentaacetic | 1:1 to 1:10 |
| acid | |
| Adenosine diphosphate (ADP) | 3:1 to 30:1 |
| 3-(2-Pyridyl)-5,6-bis(5- | 3:1 to 30:1 |
| sulfo-2-furyl)-1,2,4-triazine | |
| (ferene) | |
| Picolinic Acid | 3:1 to 30:1 |
| Citrate | 3:1 to 30:1 |
| 3-(2-Pyridyl)-5,6-diphenyl- | 3:1 to 30:1 |
| 1,2,4-triazine-4,4-disulfonic | |
| acid (ferrozine) | |
| 1,10 Phenanthroline | 3:1 to 30:1 |

### EXAMPLE 8

The following example uses the release of iron from ferritin assay to show the effect of naphthoquinones on the release of iron from ferritin during ultrasound exposure. All solutions were prepared in pH 7 acetic acid solution containing approximately 0.2 mg/mL ferritin and 1 mM ferrozine, and the concentration of naphthoquinone indicated in the table below. Solutions were sonicated at 30 W, 2 MHz, 32-34 degrees Celsius for fifteen minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 15 minutes of treatment, an aliquot was tested for the presence of the iron-ferrozine chelate via absorbance at 562 nm. The amount of iron released was determined using the control solution corrected absorbance (subtract the absorbance of the control solution from the absorbance of the ultrasound solution). The corrected absorbance was compared to a ferrozine-iron standard curve to determine the amount of iron released. The enhanced iron release due to ultrasound exposure in the presence of the naphthoquinone was compared to the amount of enhanced iron release due to ultrasound exposure in the absence of any additives as follows:

$$\text{\% activity} = \frac{\text{iron release (with additive)} - \text{iron release (without additive)}}{\text{iron release (with additive)}} \times 100$$

Results:

| | % Ultrasound |
|---|---|
| Additive | Mediated Activity |
| 18 uM 2-methyl-1,4- | 5.3% |
| naphthoquinone | |
| (menadione) | |
| 10 uM 5-hydroxy-1,4- | 93% |
| naphthoquinone | |
| (juglone) | |
| 15 uM 2-hydroxy-3-(3- | 72% |
| methyl-2-butenyl)-1,4- | |
| naphthoquinone | |
| (lapachol) | |
| 71 uM 5-hydroxy-2- | 155% |
| methyl-1,4- | |
| naphthoquinone | |
| (plumbagin) | |
| 106 uM 5,8 dihydroxy - | 185% |
| 1,4-naphthoquinone | |

### EXAMPLE 9

The following example uses the release of iron from ferritin assay to show the effect of anthraquinones on the release of iron from ferritin during ultrasound exposure. All solutions are prepared in pH 7.5 phosphate buffer containing approximately 0.2 mg/mL ferritin and 1 mM ferrozine, and the concentration of anthraquinone indicated in the table below. Solutions are sonicated at 30 W, 2 MHz, 32-34 degrees Celsius for fifteen minutes using a PZT-8 1.8 cm diameter custom transducer. The sonicated solution is placed on an orbit shaker rotating at 25 RPM to ensure even sonication of the solution while the transducer is held stationary. Control solutions are placed in a controlled temperature bath at 32-34 degrees Celsius without sonication. After 15 minutes of treatment, an aliquot is tested for the presence of the iron-ferrozine chelate via absorbance at 562 nm. The amount of iron released is determined using the control solution corrected absorbance (subtract the absorbance of the control solution from the absorbance of the ultrasound solution). The corrected absorbance is compared to a ferrozine-iron standard curve to determine the amount of iron released. The enhanced iron release due to ultrasound exposure in the presence of the anthraquinone is compared to the amount of enhanced iron release due to ultrasound exposure in the absence of any additives as follows:

$$\text{\% activity} = \frac{\text{iron release (with additive)} - \text{iron release (without additive)}}{\text{iron release (with additive)}} \times 100$$

Results:

| | % Ultrasound |
|---|---|
| Additive | Mediated Activity |
| Anthraquinone-2- | <10% |
| sulfonic acid | |
| 0.05 mM Alizarin Red S; | >50% |
| 3,4-dihydroxy-9,10- | |
| dioxo-2- | |
| anthracenesulfonic acid | |
| 0.05 mM Rhein; 9,10- | >50% |
| dihydro-4,5-dihydroxy- | |
| 9,10-dioxo-2- | |
| anthracenecarboxylic | |
| acid | |
| 0.05 mM Chrysophanol; | >50% |
| 1,8-dihydroxy-3- | |
| methylanthraquinone | |
| 0.05 mM Emodin; 6- | >50% |
| methyl-1,3,8- | |
| trihydroxyanthraquinone | |

### EXAMPLE 10

The following example uses the release of iron from ferritin assay to show the effect of additives on the release of iron from ferritin during ultrasound exposure. All solutions were prepared in pH 7 acetic acid solution containing approximately 0.2 mg/mL ferritin and 1 mM ferrozine, and the concentration of 1,4-quinone indicated in the table below. Solutions were sonicated at 30 W, 2 MHz, 32-34 degrees Celsius for fifteen minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 15 minutes of treatment, an aliquot was tested for the presence of the iron-ferrozine chelate via absorbance at 562 nm. The amount of iron released was determined using the control solution corrected absorbance (subtract the absorbance of the control solution from the absorbance of the ultrasound solution). The corrected absorbance was compared to a ferrozine-iron standard curve to determine the enhanced iron release due to ultrasound exposure. The enhanced iron release due to ultrasound exposure in the presence of the additive was compared to the amount of enhanced iron release due to ultrasound exposure in the absence of any additives as follows:

$$\text{\% activity} = \frac{\text{iron release (with additive)} - \text{iron release (without additive)}}{\text{iron release (with additive)}} \times 100$$

Results:

| | % Ultrasound Mediated |
|---|---|
| Additive | Activity |
| 1,4 benzoquinone | 0% |
| Tetrahydroxy 1,4- | 186% |
| benzoquinone | |
| (0.11 mM) | |
| DIHYDROXYFUMARATE (0.01 mM) | 72% |
| CYSTEINE (0.45 mM) | 160% |
| PENICILLAMINE (0.11 mM) | 117% |

### EXAMPLE 11

The following example uses the thiobarbituric acid-reactive substances (TBARS) assay to identify metals that enhance radical production during ultrasound. All solutions are prepared in pH 7.5 phosphate buffer containing approximately 2 mM deoxyribose, 0.01% hydrogen peroxide, 0.05 mM ferrous iron added as FeSO₄ hydrate, 0.075 mM EDTA, and the additives indicated in the table below. Solutions are sonicated at 30 W, 2 MHz, 32-34 degrees Celsius for ten minutes using a PZT-8 1.8 cm diameter custom transducer. The sonicated solution is placed on an orbit shaker rotating at 25 RPM to ensure even sonication of the solution while the transducer is held stationary. Control solutions are placed in a controlled temperature bath at 32-34 degrees Celsius without sonication. After 10 minutes of treatment, 1 mL test solution is placed in a test tube followed by 2 mL of 1% 2-thiobarbituric acid and 2 mL of 2.8% trichloroacetic acid. The test tube is sealed and heated to 90 degrees Celsius for 30 minutes and allowed to cool to room temperature for 20 minutes. The absorbance at 532 nm is measured. The enhanced radical production during ultrasound exposure is determined by comparing the amount of deoxyribose degradation that occurs in the sonicated solution versus the control solution using the following equation:

$$\% activity = \frac{{Abs}_{532} sonicated solution - {Abs}_{532} control solution}{{Abs}_{532} control solution} \times 100$$

Results:

| | % Ultrasound |
|---|---|
| | Mediated |
| | Activity vs |
| Additive | Control |
| No additive | <20% |
| Gossypol (0.075 mM) | >100% |
| Quercetin (0.075 mM) | >100% |
| Myricetin (0.075 mM) | >100% |

Addition of 0.075 mM ascorbate or cysteine significantly increased radical production in the sonicated versus control solution.

### EXAMPLE 12

The following example uses the thiobarbituric acid-reactive substances (TBARS) assay to identify anti tumor antibiotics that enhance radical production during ultrasound. All solutions are prepared in pH 7.5 phosphate buffer containing approximately 2 mM deoxyribose, 0.01% hydrogen peroxide, 0.005 mM ferrous iron, 0.005 mM ferric iron, and the additives indicated in the table below. Solutions are sonicated at 30 W, 2 MHz, 32-34 degrees Celsius for fifteen minutes using a PZT-8 1.8 cm diameter custom transducer. The sonicated solution is placed on an orbit shaker rotating at 25 RPM to ensure even sonication of the solution while the transducer is held stationary. Control solutions are placed in a controlled temperature bath at 32-34 degrees Celsius without sonication. After 15 minutes of treatment, 1 mL test solution was placed in a test tube followed by 2 mL of 1% 2-thiobarbituric acid and 2 mL of 2.8% trichloroacetic acid. The test tube is sealed and heated to 90 degrees Celsius for 30 minutes and allowed to cool to room temperature for 20 minutes. The absorbance at 532 nm is measured. The enhanced radical production during ultrasound exposure is determined by comparing the amount of deoxyribose degradation that occurs in the sonicated solution versus the control solution using the following equation:

$$\% activity = \frac{{Abs}_{532} sonicated solution - {Abs}_{532} control solution}{{Abs}_{532} control solution} \times 100$$

Results:

| | % Ultrasound |
|---|---|
| | Mediated Activity vs |
| Additive | Control |
| No additive | <20% |
| Mitomycin C, 0.025 mM | >100% |
| Streptonigrin, 0.025 mM | >100% |
| Mithramycin, 0.025 mM | >100% |
| Olivomycin, 0.025 mM | >100% |
| Chromomycin, 0.025 mM | >100% |
| Carminic acid, 0.025 mM | >100% |
| Daunomycin, 0.1 mM | >100% |
| Epirubicin, 0.1 mM | >100% |

### EXAMPLE 13

The following example uses the thiobarbituric acid-reactive substances (TBARS) assay to identify existing sonodynamic agents that exhibit enhanced radical production during ultrasound exposure in the presence of a metal. All solutions were prepared in pH 7.5 phosphate buffer containing approximately 2 mM deoxyribose, 0.01% hydrogen peroxide, 0.025 mM ferrous iron, 0.025 mM ferric iron, and the additives indicated in the table below. Solutions were sonicated at 30 W, 2 MHz, 32-34 degrees Celsius for ten minutes using a PZT-8 1.8 cm diameter custom transducer. The sonicated solution was placed on an orbit shaker rotating at 25 RPM to ensure even sonication of the solution while the transducer was held stationary. Control solutions were placed in a controlled temperature bath at 32-34 degrees Celsius without sonication. After 10 minutes of treatment, 1 mL test solution was placed in a test tube followed by 2 mL of 1% 2-thiobarbituric acid and 2 mL of 2.8% trichloroacetic acid. The test tube was sealed and heated to 90 degrees Celsius for 30 minutes and allowed to cool to room temperature for 20 minutes. The absorbance at 532 nm was measured. The enhanced radical production during ultrasound exposure was determined by comparing the amount of deoxyribose degradation that occurs in the sonicated solution versus the control solution using the following equation:

$$\% activity = \frac{{Abs}_{532} sonicated solution - {Abs}_{532} control solution}{{Abs}_{532} control solution} \times 100$$

Results:

| | % Ultrasound |
|---|---|
| | Mediated Activity |
| Additive | vs Control |
| No additive | 19% |
| Hematoporphyrin (0.027 mM) | 24% |
| Rose Bengal (0.028 mM) | 28% |
| Adriamycin (0.029 mM) | 29% |
| Tetracycline (0.030 mM) | 51% |

### EXAMPLE 14

The following example uses the thiobarbituric acid-reactive substances (TBARS) assay to identify existing sonodynamic agents that exhibit enhanced radical production during ultrasound exposure in the presence of a metal. All solutions are prepared in pH 7.5 phosphate buffer containing approximately 2 mM deoxyribose, 0.01% hydrogen peroxide, 0.025 mM ferrous iron, 0.025 mM ferric iron, and the additives indicated in the table below. Solutions are sonicated at 30 W, 2 MHz, 32-34 degrees Celsius for ten minutes using a PZT-8 1.8 cm diameter custom transducer. The sonicated solution is placed on an orbit shaker rotating at 25 RPM to ensure even sonication of the solution while the transducer is held stationary. Control solutions are placed in a controlled temperature bath at 32-34 degrees Celsius without sonication. After 10 minutes of treatment, 1 mL test solution is placed in a test tube followed by 2 mL of 1% 2-thiobarbituric acid and 2 mL of 2.8% trichloroacetic acid. The test tube is sealed and heated to 90 degrees Celsius for 30 minutes and allowed to cool to room temperature for 20 minutes. The absorbance at 532 nm is measured. The enhanced radical production during ultrasound exposure is determined by comparing the amount of deoxyribose degradation that occurs in the sonicated solution versus the control solution using the following equation:

$$\% activity = \frac{{Abs}_{532} sonicated solution - {Abs}_{532} control solution}{{Abs}_{532} control solution} \times 100$$

Results:

| | % Ultrasound |
|---|---|
| | Mediated Activity |
| Additive | vs Control |
| No additive | 20% |
| Hypocrellin A (0.025 mM) | >30% |
| Hypericin (0.025 mM) | >30% |
| Iron(III) phthalocyanine- | >30% |
| 4,4′,4″,4″′-tetrasulfonic | |
| acid (0.025 uM) | |

Metal toxicity occurs by three mechanisms. First, metals propagate free radical chain reactions on which continued radical production depends. Second, traces of metals are required for Fenton type reactions. Third, metals provide for site-specific production of active species, as in binding to DNA to provide centers for repeated generation of ferryl species or hydroxyl radicals. Therefore, it is believed that the compositions of the present invention may be effective because of one of these mechanisms or a combination of mechanisms.

The foregoing description of the specific embodiments will so fully reveal the general nature of the invention that others can, by applying current knowledge, readily modify and/or adapt for various applications such specific embodiments without undue experimentation and without departing from the generic concept. Therefore, such adaptations and modifications should and are intended to be comprehended within the meaning and range of equivalents of the disclosed embodiments. It is to be understood that the phraseology or terminology employed herein is for the purpose of description and not of limitation. The means and materials for carrying our various disclosed functions may take a variety of alternative forms without departing from the invention. Thus, the expressions “means to” and “means for” as may be found in the specification above and/or in the claims below, followed by a functional statement, are intended to define and cover whatever structural, physical, chemical, or electrical element or structures which may now or in the future exist for carrying out the recited function, whether or not precisely equivalent to the embodiment or embodiments disclosed in the specification above; and it is intended that such expressions be given their broadest interpretation.

All references cited herein are incorporated by reference.

### References

Babbs, C. F. Free radicals and the etiology of colon cancer. Free Radicals Biology & Medicine. Vol. 8 pp191-200 (1990).

Canada, A. The production of reactive oxygen species by dietart flavonols. Free Radical Biology & Medicine. Vol 9. pp441-449 (1990).

Cassanelli, S. Sulfide is an efficient iron releasing agent for mammalian ferritins. Biochimica et Biophysica Acta. Vol 1547 pp174-182 (2001).

Chen, F. One-electron reduction of chromium (VI) by alpha-lipoic acid and related hydroxyl radical generation, dG hydroxylation and nuclear transcription factor kB activation. Archives of Biochemistry and Biophysics. Vol 338 pp 165-172 (1997).

Diez, L. High performance liquid chromatographic assay of hydroxyl free radical using salicylic acid hydroxylation during in vitro experiment sinvolving thiols. Journal of Chromatography B, Vol 763 pp185-193 (2001).

Dognin, J. Mobilisation of iron from ferritin fractions of defined iron content by biological reductants. FEBS Letters. Vol 54. pp234-236 (1975).

Donlin, M. J. Analysis of iron in ferritin, the iron-storage protein: a general chemistry experiment. J. Chem. Edu. 75, 437-441 (1998).

Graf, E. Iron-catalyzed hydroxyl radical formation. The Journal of Biological Chemistry. Vol 259, No. 6, pp 3620-3624 (1984).

Gutteridge J M. Free radical damage to deoxyribose by anthracycline, aureolic acid and aminoquinone antitumour antibiotics. An essential requirement for iron, semiquinones and hydrogen peroxide. Biochem Pharmacol. Vol. 34(23) pp 4099-103 (1985).

Gutteridge J M Mitomycin C-induced deoxyribose degradation inhibited by superoxide dismutase. A reaction involving iron, hydroxyl and semiquinone radicals. FEBS Lett. Vol. 167(1) pp 37-41 (1984).

Gutteridge J M, Carminic acid-promoted oxygen radical damage to lipid and carbohydrate. Food Addit Contam. Vol 3(4) pp 289-93 (1986).

Gutteridge, J. M. C. Damage to biological molecules by iron and copper complexes. Lipofuscin. Pp69-82 (1987).

Halliwell, B. The deoxyribose method: a simple test-tube assay for determination of rate constants for reactions of hydroxyl radicals. Analytical Biochemistry. Vol 165 pp 215-219 (1987).

Hristov, P. Lipid peroxidation induced by ultrasonication in Ehrlich ascitic tumor cells. Cancer Letters. Vol. 121 pp 7-10 (1997).

Inoue, S. Hydroxyl radical production and human DNA damage induced by ferric nitrilotriacetate and hydrogen peroxide. Cancer Research. Vol. 47 pp 6522-6527 (1987).

Kagedal K, Bironaite D, Ollinger K. Anthraquinone cytotoxicity and apoptosis in primary cultures of rat hepatocytes. Free Radic Res.Vol. 31(5) pp 419-28 (1999).

Laughton, M. Antioxidant and prooxidant actions of the plant phenolics quercetin, gossypol, and myricetin. Biochemical Pharmacology. Vol. 38. pp2859-2865 (1989).

Lee H Z.Effects and mechanisms of emodin on cell death in human lung squamous cell carcinoma. Br J. Pharmacol. Vol. 134(1) pp 11-20 (2001).

Lee H Z, Hsu S L, Liu M C, Wu C H.Effects and mechanisms of aloe-emodin on cell death in human lung squamous cell carcinoma. Eur J. Pharmacol. Vol 431(3) pp 287-95 (2001).

Lawson, R. C., Sonochemistry of quinones in argon-saturated aqueous solutions: enhanced cytochrome c reduction. Chem. Res. Toxicol. Vol 12. pp 850-854 (1999).

Lindqvist, C. Generation of hydroxyl radicals by the antiviral compound phosphoneformic acid (foscarnet). Pharmacology & Toxicology. Vol 89 pp 49-55 (2001).

Mascio, P. D. DNA damage by 5-aminoevulinic and 4,5 dioxovaleric acids in the presence of ferritin. Archives of Biochemistry and Biophysics. Vol 373 pp 368-374 (2000).

Morier-Teissier E. Free radical production and DNA cleavage by copper chelating peptide-anthraquinones. Anticancer Drug Des. Vol. 5(3) pp 291-305 (1990).

Muller K, Gurster D. Hydroxyl radical damage to DNA sugar and model membranes induced by anthralin (dithranol). Biochem Pharmacol. Vol 46(10) pp 1695-704 (1993).

Ou, Z. Metal ions affect on the photodynamic actions of cyclodextrin-modified hypocrellin. Cancer Letters. pp 206-207 (2002).

Quinlan, G. Hydroxy radical generation by the tetracycline antibiotics with free radical damage to DNA, lipids, and carbohydrates in the presence of iron and copper salts. Free Radical Biology & Medicine. Vol 5 pp341-348 (1998).

Ryter, S. W. The heme synthesis and degradation pathways: role in oxidant sensitivity. Free Radical Biology & Medicine. Vol 28 pp289-309 (2000).

Schneider, J. E. Ascorbate/iron mediation of hydroxyl free radical damage to PBR322 plasmid DNA. Free Radical Biology & Medicine. Vol 5 pp287-295 (1988).

Stadtman, E. R. Fenton chemistry. The Journal of Biological Chemistry. Vol 266 pp 17201-17211 (1991).

Thomas, C. The hydrolysis product of ICRF-187 promotes iron-catalyzed hydroxyl radical production via the Fenton reaction. Biochemical Pharmacology. Vol. 45, No. 10, pp 1967-1972 (1993).

Thomas, C. E. Release of iron from ferritin by cardiotoxic anthracycline antibiotics. Arch. Biochem. Biophys. 248, 684-689 (1986).

Tonetti, M. Enhanced formation of reactive species from cis-diammine-(1,1-cyclobutanedicarboxylato)-platinum(II) (carboplatin) in the presence of oxygen free radicals. Biochemical Pharmacology. Vol 46 pp 1377-1383 (1993).

Toyokuni, S. Induction of oxidative single and double strand breaks in DNA by ferric citrate. Free Radical Biology & Medicine. Vol 15 pp 117-123 (1993).

What is claimed is:

1. A sonodynamic composition comprising a sonodynamic agent and at least one metal.

2. The sonodynamic composition according to claim 1 wherein the metal is selected from the group consisting of transition metals, lanthamides, and actinides.

3. The sonodynamic composition according to claim 2 wherein the metal is in a form selected from the group consisting of free metal ions, inorganic metal salts, organic metal salts, metal oxides, metal hydroxides, metal sulfides, coordination compounds, chelates, and clathrates.

4. A photodynamic composition comprising a photodynamic agent and at least one metal.

5. The photodynamic composition according to claim 4 wherein the metal is selected from the group consisting of transition metals, lanthamides, and actinides.

6. The photodynamic composition according to claim 5 wherein the metal is in a form selected from the group consisting of free metal ions, inorganic metal salts, organic metal salts, metal oxides, metal hydroxides, metal sulfides, coordination compounds, chelates, and clathrates.

7. A method for enhancing the formation of free radicals comprising subjecting the combination of a photodynamic agent and a metal to light waves.

8. The method according to claim 7 wherein the metal is selected from the group consisting of transition metals, lanthamides, and actinide.

9. The method according to claim 8 wherein the metal is in a form selected from the group consisting of free metal ions, inorganic metal salts, organic metal salts, metal oxides, metal hydroxide, metal sulfides, coordination compounds, chelates, and clathrates.

10. The method according to claim 7 wherein the combination of photodynamic agent and a metal further includes a compound that produces a bicarbonate.

11. A method for enhancing the formation of free radicals comprising subjecting the combination of a sonodynamic agent and a metal to sound waves.

12. The method according to claim 11 wherein the metal is selected from the group consisting of transition metals, lanthamides, and actinide.

13. The method according to claim 12 wherein the metal is in a form selected from the group consisting of free metal ions, inorganic metal salts, organic metal salts, metal oxides, metal hydroxide, metal sulfides, coordination compounds, chelates, and clathrates.

14. The method according to claim 12 wherein the combination of sonodynamic agent and a metal further includes a compound that produces a bicarbonate.

15. A method for treating a mammal by photodynamic therapy or sonodynamic therapy comprising administering a photodynamic agent or a sonodynamic agent and a metal to the mammal and exposing the mammal to light waves or to sound waves.

16. The method according to claim 15 wherein the metal is administered simultaneously with the photodynamic agent.

17. The method according to claim 15 wherein the metal is administered prior to administration of the photodynamic agent or the sonodynamic agent.

18. The method according to claim 15 wherein the metal is administered after administration of the photodynamic agent or the sonodynamic agent.

19. The method according to claim 15 wherein the metal is selected from the group consisting of transition metals, lanthamides, and actinides.

20. The method according to claim 19 wherein the metal is in a form selected from the group consisting of free metal ions, inorganic metal salts, organic metal salts, metal oxides, metal hydroxides, metal sulfides, coordination compounds, chelates and clathrates.

21. The method according to claim 15 wherein the mammal is also administered an activator for a photodynamic agent or a sonodynamic agent, said activator selected from the group consisting of transition metals, chelants, a compound that exhibits increased thiobarbituric acid resistance in the presence of a metal and hydrogen peroxide, a reductant, a macrophage/neutrophil stimulator, and compounds with prooxidant activity.

22. A method for enhancing the formation of free radicals comprising subjecting the combination of a sonodynamic agent and an activator for the sonodynamic agent to sound waves.

23. The method according to claim 22 wherein the activator is selected from the group consisting of iron, reductants, chelants, and mixtures thereof.

24. The method according to claim 15 wherein the sonodynamic agent is a quinone compound.

25. The method according to claim 24 wherein the quinone compound is generated from an azo dye upon exposure to ultrasound.

26. The method according to claim 24 wherein the quinone compound is an anthraquinone.

27. The method according to claim 23 wherein the activator comprises a mixture of iron, a reductant, and a chelant.

28. A method for generating free radicals comprising subjecting aqueous ferrous iron in the presence of a reducing agent to ultrasound.

29. The method according to claim 27 wherein the reducing agent is oxidized ascorbic acid.

30. The method according to claim 29 wherein the iron is in the form of ferritin.

31. The method according to claim 15 wherein the activator is a combination of iron and ascorbic acid and at least one of the activators is encapsulated in a material which is destroyed by contact with ultrasound.

32. A sonodynamic composition comprising a sonodynamic agent, at least one metal, and at least one compound that enhances free radical production.

33. The sonodynamic composition according to claim 32 further including at least one compound that alters cell membrane permeability.

34. The sonodynamic composition according to claim 33 further including a compound that exhibits iron release from biological compounds containing iron in the presence of ultrasound.

35. A photodynamic composition comprising a photodynamic agent, at least one metal, and at least one compound that enhances free radical production.

36. The method according to claim 7 wherein the combination of a photodynamic agent and a metal further includes are least one member of the group consisting of compounds that show increased thiobarbituric acid reactive substances (TBARS) in the presence of a metal and hydrogen peroxide, compounds that exhibit iron release from biological compounds containing iron in the presence of ultrasound, chelants which produce free radical production when exposed to ultrasound including aminocarboxylates and their salts, derivatives, isomers, polymers, and iron coordination compounds, reducing agents, chelants that have available a coordination site that is free or occupied by an easily displaceable ligand, tartaric acid, glucoheptonic acid, glycolic acid, 2-hydroxyacetic acid; 2-hydroxypropanoic acid; 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxypropanoic acid; 2,3-dihydroxypropanoic acid; 2,3,4-trihydroxybutanoic acid; 2,3,4,5-tetrahydroxypentanoic acid; 2,3,4,5,6-pentahydroxyhexanoic acid; 2-hydroxydodecanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid; isocitric acid; agaricic acid; quinic acid; glucuronic acid; glucuronolactone; galacturonic acid; galacturonolactone; uronic acids; uronolactones; dihydroascorbic acid; dihydroxytartaric acid; tropic acid; ribonolactone; gluconolactone; galactonolactone; gulonolactone; mannonolactone; ribonic acid; gluconic acid; citramalic acid; pyruvic acid; hydroxypyruvic acid; hydroxypyruvic acid phosphate; methylpyruvate; ethyl pyruvate; propyl pyruvate; isopropyl pyruvate; phenyl pyruvic acid; methyl phenyl pyruvate; ethyl phenyl pyruvate; propyl phenyl pyruvate; formyl formic acid; methyl formyl formate; ethyl formyl formate; propyl formyl formate; benzoyl formic acid; methyl benzoyl formate; ethyl benzoyl formate; propyl benzoyl formate; 4-hydroxybenzoyl formic acid; 4-hydroxyphenyl pyruvic acid; 2-hydroxyphenyl pyruvic acid, chelants which increase free radical production when exposed to ultrasound and a metal, including adenosine diphosphate (ADP), adenosine triphosphate (ATP) and guanosine triphosphate (GTP), reducing agents including ascorbic acid, 1,4-naphthoquinone derivatives, 1,4 benzoquinone derivatives, and 1,4-anthraquinone derivatives and/or thiols, phosphonoformic acid, phosphonoacetic acid, and pyrophosphate, biological chelants including ADP, ATP, and GTP, tetracycline antibiotics and their derivatives, salts, and polymers thereof, hydroxy-1,4-naphthoquinones, their derivatives, isomers, metal coordination compounds, salts, and polymers thereof, including 1,4-naphthalenedione, 2,3-dihydroxy; 1,4-naphthalenedione, 2,5,8-trihydroxy; 1,4-naphthalenedione, 2-hydroxy; 1,4-naphthalenedione, 2-hydroxy-3-(3-methylbutyl); 1,4-naphthalenedione, 2-hydroxy-3-methyl; 1,4-naphthalenedione, 5,8-dihydroxy-2-methyl; alkannin; alkannin dimethylacrylate; aristolindiquinone, chleone A, droserone; isodiospyrin; naphthazarin; tricrozarin A, actinorhodine, euclein, and atovaquone; hydroxylated 1,4-benzoquinones, their derivatives, isomers, metal coordination compounds, salts, and polymers thereof; hydroxylated anthraquinones, their derivatives, isomers, metal coordination compounds, salts, and polymers; hydroxylated anthraquinones and their derivatives, including alizarin, aloe-emodin, anthragallol, aurantio-obtusin, barbaloin, cascaroside A, cassiamin C, 7-chloroemodi, chrysazin, chryso-obtusin, chrysophanic acid 9-anthrone, digiferrugineol, 1,4-dihydroxy-2-methylanthraquinone, frangulin A, frangulin B, lucidin, morindone, norobtusifolin, obtusifolin, physcion, pseudopurpurin, purpurin, danthron, and rubiadin; flavonoids including kaempferol, quercetin, and myricetin and sesquiterpenes including gossypol and feralin, cacetin, apigenin, biochanin-A, daidzein, equol, flavanone, flavone, formononetin, genistin, glabranin, liquiritigenin, luteolin, miroestrol, naringenin, naringin, phaseollin, phloretin, prunetin, robinin, and sophoricoside, derivatives, polymers, and glycosylated forms thereof; anti-tumor antibiotic quinoid agents including benzoquinones, mitimycins, streptonigrins, actinomycins, anthracyclines, and substituted anthraquinones; thiol compounds, their derivatives, and polymers including cysteinylglycine, cysteamine, thioglycollate and glutathione, Captopril, Pyritinol (pyridoxine disulfide), Thiopronine, Piroxicam, Thiamazole, 5-Thiopyridoxine, Gold sodium thiomalate, bucillamine, 1-(mercaptomethyl)-7,7-dimethylbicyclo[2.2.1]heptan-2-one; 1,2,3-benzotriazine-4(3H)-thione; 1,2-benzisothiazole-3(2H)-thione-1,1-dioxide;1,2-dihydro-3H-1,2,4-triazole-3-thione; 1,2-dihydro-3H-1,2,4-triazole-3-thione and derivatives; 1,2-dihydro-4,5-dimethyl-2H-imidazole-2-thione; 1,3-dihydro-1-methyl-2H-imidazole-2-thione; 1,3-dihydro-2H-naphth[2,3-d]imidazole-2-thione; 1,3-dihydro-4,5-diphenyl-2H-imidazole-2-thione; 1,4-benzoxazepine-5(4H)-thione; 1,4-dihydro-5H-tetrazole-5-thione and derivatives; 1,5-dihydro-4H-pyrazolo[3,4-d]pyrimidine-4-thione; 1,5-dihydro-6H-imidazo[4,5-c]pyridazine-6-thione; 1,7-dihydro-6H-purine-6-thione; 1-adamantanethiol; 2(1H)-benzimidazolinethione; 2,4-diamino-6-mercapto-1,3,5-triazine; 2,4-dimethylbenzenethiol; 2,5-dimethylbenzenethiol; 2,6-dimethylbenzenethiol; 2-adamantanethiol; 2-amino-1,7-dihydro-6H-purine-6-thione; 2H-1,4-benzothiazine-3(4H)-thione; 2-imidazolidinethione; 2-Isopropyl-3-methylbenzenethiol; 2-isopropyl-4-methylbenzenethiol; 2-isopropyl-5-methylbenzenethiol; 2-mercapto-4H-1-benzopyran-4-thione; 2-mercapto-5-methyl-1,3,4-thiadiazole; 2-mercapto-5-nitrobenzimidazole; 2-mercaptothiazoline; 2-methyl-1-propenethiol; 2-methylene-1,3-propanedithiol; 2-propene-1-thiol; 3,4-dihydro-4,4,6-trimethyl-1-(4-phenyl-2-thiazolyl)-2(1H)-pyrimidinethione; 3,4-dihydro-4,4,6-trimethyl-2(1H)-pyrimidinethione; 3-amino-5-mercapto-1H-1,2,4-triazole; 3-bromo-1-adamantanethiol; 3-mercapto-5-methyl-1,2,4-triazole and derivatives; 3-mercaptocyclohexanone and derivatives; 3-quinuclidinethiol; 3-thio-9,10-secocholesta-5,7,10(19)-triene; 4-amino-2,4-dihydro-5-phenyl-3H-1,2,4-triazole-3-thione; 4-amino-3-hydrazino-5-mercapto-1,2,4-triazole; 4-benzocyclobutenethiol; 4-biphenylthiol; 4-Isopropyl-2-methylbenzenethiol; 5,6-dichloro-2-mercapto-1H-indole; 5′-amino-2′,3,3′,4-tetrahydro-4,4,6-trimethyl-2,21-dithioxo[1(2H),4′-bipyrimidin]-6′(1′H)-one; 5-isopropyl-2-methylbenzenethiol; 5-mercapto-3-methyl-1,2,4-thiadiazole; 6-amino-2-mercaptopurine; 6-thioinosine; 7-(mercaptomethyl)-1,7-dimethylbicyclo[2.2.1]heptan-2-one; 7-mercapto-3H-1,2,3-triazolo[4,5-d]pyrimidine; Azothiopyrine; benzo[c]thiophene-1(3H)-thione; bis(1-methylethyl)carbamothioic acid S-(2,3,3-trichloro-2-propenyl) ester; Caesium [2,6-bis(2,4,6-triisopropylphenyl)phenyl]thiolate; (3β)-cholest-5-ene-3-thiol; Cyclohexanethione; Lithium [2,6-bis(2,4,6-triisopropylphenyl)phenyl]thiolate; naphtho[1,2-d]thiazole-2(1H)-thione; naphtho[2,1-d]thiazole-2(3H)-thione; phenylmethanethiol; Potassium [2,6-bis(2,4,6-triisopropylphenyl)phenyl]thiolate; Rubidium [2,6-bis(2,4,6-triisopropylphenyl)phenyl]thiolate; Sodium [2,6-bis(2,4,6-triisopropylphenyl)phenyl]thiolatedrugs classified as penicillins, cephalosporins, and piroxicam; reducing agents including sodium sulfide and sodium sulfite.
